(19) United States
(12) Patent Application Publication
(10) Pub. No.: US 2026/0347392 A1
(43) Pub. Date: Nov. 5, 2026

(54) SYSTEMS AND METHODS FOR PRODUCING THERAPEUTIC PROTEINS USING SINGLE PASS TANGENTIAL FLOW FILTRATION FOR IN-LINE CONCENTRATION AND VOLUME REDUCTION OF PRODUCTION POOLS

(71) Applicant: Regeneron Pharmaceuticals, Inc., Tarrytown, NY (US)

(72) Inventors: Claire Tantillo, New York, NY (US); Charles Palin, Stamford, CT (US); Christina Passno, Allendale, NJ (US); Christopher Cowan, Briarcliff Manor, NY (US)

(21) Appl. No.: 19/348,183

(22) Filed: Oct. 2, 2025

Related U.S. Application Data
(60) Provisional application No. 63/702,695, filed on Oct. 3, 2024.
(60) Provisional application No. 63/768,397, filed on Mar. 7, 2025.

(51) Int. Cl. C07K 1/34; C07K 1/18; C07K 1/20; C07K 1/22; C07K 16/00

(52) U.S. Cl. CPC C07K 1/34; C07K 1/18; C07K 1/20; C07K 1/22; C07K 16/00

(57) ABSTRACT

The inventions provide improved systems and methods for inline concentrating and reducing intermediate process pools to mitigate plant fit challenges associated with high-titer protein purification process. The inventions also provide methods for concentrating and reducing VI pool volumes by integrating columns of single pass tangential flow filtration (SPTFF) system with viral inactivation pool filtration (VIPF) in protein purification process. Therapeutic proteins purified by the methods also are provided.

This application claims priority to U.S. Application Ser. No. 63/702,695, filed Oct. 3, 2024; and U.S. Application Ser. No. 63/768,397, filed Mar. 7, 2025. These applications are incorporated by reference in their entirety.

### FIELD OF THE INVENTIONS

The present inventions provide improved systems and methods for in-line concentrating and reducing intermediate pools to mitigate plant fit challenges associated with high-titer therapeutic protein purification processes. The inventions also provide systems and methods for concentrating and reducing Viral Inactivated (VI) pools by incorporating single pass tangential flow filtration (SPTFF) techniques in the train of protein purification process.

### BACKGROUND OF THE INVENTIONS

Advances in cell line technologies, cell culture media composition, and bioreactor operating parameters have resulted in increased protein (such as antibody including monoclonal antibody) titers in the bioreactor from less than 1 g/L (gram/liter) to greater than 10 g/L over the last 20 years. These high-titer processes provide increased productivity, allowing industries to meet the growing demand for various unmet medical needs. However, high-titer processes also pose various facility fit challenges. Processes with greater than 5 g/L bioreactor titer can fail to fit the volume in intermediate holding tanks during various downstream purification unit operations. One approach to resolve this challenge is to split a bioreactor into multiple purification trains. Such split batch processing significantly increases processing time, increases the cost of goods (COGS), and requires more space in the manufacturing suites.

Therefore, a volume reduction approach is needed to fit retentate volume in the production tanks, typically intermediate tanks, during the polishing steps. The inventions disclosed herein provide solutions to mitigate volume-related challenges by concentrating the intermediate process pools in-line by optimized incorporation of single pass tangential flow filtration (SPTFF).

### SUMMARY OF THE INVENTIONS

The inventions advantageously provide improved methods of purifying a therapeutic protein from a high-titer cell culture, wherein the methods comprise the steps of: (a) subjecting the high-titer cell culture to affinity capture chromatography (ACX) and viral inactivation (VI) to generate a VI pool; (b) subjecting the VI pool to VI pool filtration (VIPF) system connected to a single pass tangential flow filtration (SPTFF) system, thereby concentrating the pool volume, and generating a VIPF-SPTFF pool; (c) subjecting VIPF-SPTFF pool to a chromatography 1 column and then a chromatography 2 column to generate a pool to load on a virus retentive filtration (VRF) system to generate a VRF pool, wherein the chromatography 1 column and the chromatography 2 column are the same or different, preferably different; and (d) subjecting the VRF pool to ultrafiltration and/or diafiltration (UF/DF), thereby obtaining the therapeutic protein from the high-titer cell culture. Step (c) can further comprise a chromatography 3 column that is the same or different (preferably different) from chromatography 1 columns and/or 2, and downstream from the chromatography 1 column and the chromatography 2 column, wherein the pool from the chromatography 3 column can be loaded on the VRF system to generate the VRF pool. Chromatography 1 column can be selected from the group consisting of an anion exchange chromatography (AEX) column, a cation exchange chromatography (CEX) column, a hydrophobic interaction chromatography (HIC) column. Chromatography 2 column is selected from the group consisting of an AEX column, a CEX column, and a HIC column. Chromatography 3 column can be selected from the group consisting of an AEX column, a CEX column, and a HIC column. Chromatography columns can contain chromatographic media in the form of monoliths, beads, resins, gels, and membranes, for example. Additional chromatography columns are optional. The inventions also advantageously provide therapeutic proteins made by these methods.

The inventions also advantageously provide improved methods of purifying therapeutic proteins from a high-titer cell culture, wherein the methods comprise the steps of: (a) subjecting the high-titer cell culture to affinity capture chromatography (ACX) and viral inactivation (VI) to generate a VI pool; (b) subjecting the VI pool to VI pool filtration (VIPF) system serial connected to a single pass tangential flow filtration (SPTFF) system, thereby concentrating the pool volume and generating a VIPF-SPTFF pool; (c) subjecting the VIPF-SPTFF pool to anion exchange chromatography (AEX) column to generate an AEX pool; (d) subjecting the AEX pool to a hydrophobic interaction chromatography (HIC) column to generate an HIC pool; (e) subjecting the HIC pool to a virus retentive filtration (VRF) system to generate a VRF pool; and (f) subjecting the VRF pool to ultrafiltration and/or diafiltration (UF/DF), thereby obtaining the therapeutic protein from the high-titer cell culture. According to the inventions, concentrating the VI pool reduces the volume of VIPF-SPTFF pool to 10% to 90% of the VI pool volume. The high-titer bioreactor protein concentration can be about 1 g/L to about 25 g/L; The protein concentration in the pool can be at least 5 g/L to about 50 g/L. The SPTFF system of the inventions can comprise 1 to 20 SPTFF (or more) capsules, and the SPTFF capsules can be connected in a serial and/or a parallel configuration. The cell culture can be a mammalian cell culture. For example, the mammalian cell is a CHO, HeLa, BHK or HEK293 cell, or any variant thereof. The therapeutic protein can be an antibody, such as a monoclonal antibody.

The flow rates can be the same across the VIPF filtration and the SPTFF system. Concentrating the pool volume and volume reduction by SPTFF can be controlled by controlling the overall filtration and SPTFF process flow rate or by SPTFF back pressure.

The inventions also provide therapeutic proteins obtained from a high-titer cell culture, wherein the therapeutic protein is purified by methods comprising the steps of: (a) subjecting the high-titer cell culture to affinity capture chromatography and viral inactivation (VI) to generate a VI pool; (b) subjecting the VI pool to VI pool filtration (VIPF) system serial connected to a single pass tangential flow filtration (SPTFF) system, thereby concentrating the pool volume and generating a VIPF-SPTFF pool; (c) subjecting the VIPF-SPTFF pool to anion exchange chromatography (AEX) column to generate an AEX pool; (d) subjecting the AEX pool to a hydrophobic interaction chromatography (HIC) pool to generate an HIC pool; (e) subjecting the HIC pool to a virus retentive filtration (VRF) system to generate a VRF pool; and (f) subjecting the VRF pool to ultrafiltration and/or diafiltration (UF/DF), thereby obtaining the therapeutic protein from the high-titer cell culture. According to the inventions, concentrating the VI pool reduces the volume of VIPF-SPTFF pool to at least 10% to 90% of the VI pool volume. The high-titer bioreactor protein concentration can be about 1 g/L to about 25 g/L; The protein concentration in the pool can be 5 g/L to about 50 g/L. The SPTFF system of the inventions comprises 1 to 20 (or more) SPTFF capsules, and the SPTFF capsules are connected in a serial and/or parallel configuration. The cell culture can be a mammalian cell culture, and the mammalian cell can be a CHO, HeLa, BHK or HEK293 cell, or any variant thereof. The therapeutic protein can be an antibody, such as a monoclonal antibody (mAb). The flow rates can be the same across the ACX, VIPF filtration and the SPTFF system. The concentrating the pool volume and volume reduction by SPTFF can be controlled by controlling the overall filtration and SPTFF process flow rate or by SPTFF back pressure.

The inventions also provide methods for in-line concentration and volume reduction of the intermediate process pools through SPTFF, wherein the methods comprise the steps of a normal flow filtration process connected in series to SPTFF; wherein flow rates can be the same across the filtration process and the SPTFF process, and wherein the concentration and volume reduction by SPTFF are controlled by controlling the overall filtration and SPTFF process flow rate or by SPTFF back pressure. The normal flow filtration can include affinity capture chromatography and VI pool filtration (VIPF) system. The filtration process allows removal of various impurities, including host cell proteins, through adsorptive interactions. The final feed concentration can be monitored by Raman Spectroscopy (RS), which can be used as feedback control.

The inventions also provide therapeutic protein obtained from a high-titer cell culture by any of the methods disclosed herein. The inventions also provide systems and purification trains for producing the therapeutic proteins according to the methods set forth above.

### BRIEF DESCRIPTION OF THE FIGURES

FIGS. 1A and 1B are graphs concerning production plants containing 2,000 L (FIG. 1A) and 10,000 L (FIG. 1B) bioreactors showing the pool volumes following the hydrophobic interaction chromatography (HIC) step for a monoclonal antibody (mAb) with bioreactor titer of 12 g/L with SPTFF and without SPTFF.

FIGS. 2A and 2B schematically depict exemplary high-titer purification systems and processes incorporating an SPTFF system for the production and purification of therapeutic proteins for formulation. FIG. 2A depicts pools following chromatography 1 and chromatography 2. FIG. 2B depicts AEX (chromatography 1) and HIC chromatographs (chromatography 2) following SPTFF. Although not depicted, chromatography 3 columns also can be employed according to the inventions.

FIG. 3 depicts Millipore Sigma Pellicon® Capsule configurations (series configuration and parallel configuration) for SPTFF.

FIGS. 4A and 4B show Feed flux excursion experiments (FIG. 4A) performed using the Millipore Sigma Pellicon® SPTFF capsules. Experiment set-up is illustrated in FIG. 4B.

FIG. 5 shows the results of experiments that the SPTFF pool protein concentration (and the percentage conversion) can be varied by changing feed flux at a fixed back pressure of about 8 psi.

FIG. 6 is a graph depicting the feed concentration at T₀ (clear circle) and the pool protein concentration changes over the processing time.

FIGS. 7A to 7E show results of the VIPF+SPTFF process performance and product qualities for the two VIPF platform processes. The comparison with the VIPF-only process showed a reduction in the host cell proteins (HCPs) (FIG. 7A), an increase in the high molecular weight dimer % (HMW Dimer %) (FIG. 7B), HMW higher order (%) species (FIG. 7C), a higher yield (FIG. 7D), and conversion (%) (FIG. 7E) upon the addition of SPTFF to the VIPF process. The data shown are for a mAb.

FIG. 8 shows a chart on multivariate experiments and schematic diagram for determination of the key quality attributes when the SPTFF process is combined with the VIPF unit operation using Millipore Sigma XOSP filters.

FIG. 9 schematically compares a theoretical pool tank volume distribution in a process without SPTFF having a failure rate of 6.28% to a theoretical pool tank volume distribution in a process with SPTFF having a failure rate of 0%.

FIG. 10 schematically depicts a predictive model to inform process setpoints using program-specific inputs. The figure depicts percent conversion for configuration, number of sections, feed protein concentration and feed flux.

FIG. 11 schematically depicts conversion percentage at throughput levels ranging from 0 to 100 L/m², and is based upon a VIPF filter. The shaded area is at the 95% confidence level based on the bench scale predictive model depicted in FIG. 10. Conversion was lower than 50% due to flowrates being faster than planned.

FIG. 12 is a graph depicting protein concentration by SoloVPE® (Variable Pathlength Extension) and Raman with 8 psi back pressure.

FIG. 13 is a graph depicting Raman monitoring of SPTFF concentration when back pressure is removed.

### DETAILED DESCRIPTION OF THE INVENTIONS

### Definitions

Unless defined otherwise, all technical and scientific terms used herein have the same meaning as commonly understood by one of ordinary skill in the art to which this invention belongs.

The term “about” in the context of numerical values and ranges refers to values or ranges that approximate or are close to the recited values or ranges such that the invention can perform as intended, such as having a desired rate, amount, number, density, degree, increase, decrease, percentage, ratio, value, purity, pH, concentration, presence of a form or variant, temperature or amount of time, as is apparent from the teachings contained herein. For example, “about” can signify values either above or below the stated value in a range of approximately +/−10% or more or less depending on the ability to perform. Thus, this term encompasses values beyond those simply resulting from systematic error.

“Polypeptide” or “peptide” refers to sequence(s) of amino acids covalently joined. Polypeptides include natural, semi-synthetic and synthetic proteins and protein fragments. “Polypeptide” and “protein” can be used interchangeably. Oligopeptides are considered shorter polypeptides.

“Protein of interest” or “polypeptide of interest” (POI) can have any amino acid sequence, and includes any protein, polypeptide, or peptide that is desired to be expressed. Protein types can include, but are not limited to, antibodies (and fragments and derivatives thereof), receptors, fusion proteins, agonists, antagonists, activators, inhibitors, enzymes (such as those used in enzyme replacement therapy), factors and co-factors, repressors, activators, ligands, protein hormones, therapeutic proteins, suicide proteins, structural proteins, storage proteins, transport proteins, signal proteins, neurotransmitters and contractile proteins. Derivatives, components, domains, chains, and fragments of the above also are included. The sequences can be natural, semi-synthetic or synthetic.

“Purification” in its various grammatical forms includes, but is not limited to, the use of one or more procedures such as depth filtration, tangential flow filtration, affinity capture, ionic exchange, and the like.

“Antibodies” (also referred to as “immunoglobulins”) are examples of proteins having multiple polypeptide chains and extensive post-translational modifications. The canonical immunoglobulin protein (for example, IgG) comprises four polypeptide chains—two light chains and two heavy chains. Each light chain is linked to one heavy chain via a cysteine disulfide bond, and the two heavy chains are bound to each other via two cysteine disulfide bonds. Immunoglobulins produced in mammalian systems are also glycosylated at various residues (for example, at asparagine residues) with various polysaccharides, and can differ from species to species, which may affect antigenicity for therapeutic antibodies. Butler and Spearman, “The choice of mammalian cell host and possibilities for glycosylation engineering”, Curr. Opin. Biotech. 30:107-112 (2014).

An antibody includes immunoglobulin molecules comprised of four polypeptide chains, two heavy (H) chains and two light (L) chains inter-connected by disulfide bonds. Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4 (heavy chain CDRs may be abbreviated as HCDR1, HCDR2 and HCDR3; light chain CDRs may be abbreviated as LCDR1, LCDR2 and LCDR3. The term “high affinity” antibody refers to those antibodies having a binding affinity to their target of at least 10⁻⁹ M, at least 10⁻¹⁰ M; at least 10⁻¹¹ M; or at least 10⁻¹² M, as measured by surface plasmon resonance, for example, BIACORE™ or solution-affinity ELISA.

All antibody classes, namely IgG, IgA, IgM, IgD and IgE, can be used as therapeutic proteins. IgG is a preferred class and includes subclasses IgG1 (including IgG1λ and IgG1κ), IgG2, IgG3, and IgG4. Further antibody types include a human antibody, a humanized antibody, a chimeric antibody, a monoclonal antibody (mAb), a multispecific antibody, a bispecific antibody, a trispecific antibody, an antigen binding antibody fragment, a single chain antibody, a diabody, triabody or tetrabody, a Fab fragment or a F(ab′)2 fragment, an IgD antibody, an IgE antibody, an IgM antibody, an IgG antibody, an IgG1 antibody, an IgG2 antibody, an IgG3 antibody, or an IgG4 antibody. Without limiting the applicability of the inventions, exemplary antibodies and other proteins that are amenable to the inventions are identified in Example 10.

Tangential flow filtration (TFF) includes, but is not limited to, ultrafiltration/diafiltration (UF/DF) and newer approaches such as single-pass tangential flow filtration (SPTFF). Filtration systems also can include normal flow filtration, which can include filters used for the removal of process or product-related impurities such as host cell proteins, high molecular weight species, etc. by either size based or other adsorptive interactions such as hydrophobic interaction chromatography (HIC), anion exchange chromatography (AEX), cation exchange chromatography (CEX), etc. Filtration systems also can include depth filtration, viral inactivation pool filtration (VIPF), and the like.

All numerical limits and ranges set forth herein include all numbers or values thereabout or there between of the numbers of the range or limit. The ranges and limits described herein expressly denominate and set forth all integers, decimals and fractional values defined and encompassed by the range or limit. Thus, a recitation of ranges of values herein are intended to serve as a way of referring individually to each separate value falling within the range, unless otherwise indicated herein, and each separate value is incorporated into the specification as if it were individually recited herein.

### DESCRIPTION

Protein-based therapeutic molecules are generally recombinantly produced in mammalian cell lines, such as Chinese Hamster Ovary (CHO) cell lines, in large-scale stirred tank fed-batch bioreactors. High-titer processes do not fit into current manufacturing plants that can fit only pool volumes with a bioreactor titer of no more than 5 g/L. Chromatographic purification of protein from bioreactors generally offer substantial volume reduction. However, with increased production titers, such as greater than 12 g/L bioreactor titer do not fit in intermediate holding tanks in the plant.

Fitting the high-titer processes in manufacturing plants that are typically designed for less than 5 g/L, can be challenging due to product pool volumes that exceed intermediate holding tank capacities. The current inventions provide methods to mitigate volume-related challenges by concentrating the intermediate process pools in-line by introducing single pass tangential flow filtration (SPTFF). The SPTFF provides the tools to address many volume-related facility-fit constraints associated with high-titer processes.

SPTFF utilizes a singular pass across a semi-permeable membrane to continuously concentrate a feed stream without the use of multiple pump passes or recirculations as in traditional batch ultrafiltration/diafiltration (UF/DF). A reduced feed flux, approximately five times slower than batch UF/DF, is required to provide sufficient buffer expulsion across the membrane to concentrate, for example, to a desired concentration level. If a faster flow rate is required, membrane area can be increased to shorten processing time while maintaining the same feed flux level.

The current inventions provide solutions to overcome volume-related challenges through concentrating the intermediate process pools by single pass tangential flow filtration (SPTFF). The SPTFF process with intermediate process pools for monoclonal antibody (mAb) molecules allows for ≥10% to about 90% reduction in the volume with minimal to no impact on product quality attributes. The studies disclosed herein show that SPTFF is a robust modular solution that allows for the technology to be implemented at various points in the purification process and thus provides increased manufacturing flexibility for both 2 kiloliter (kL) and 10 kL production scales.

The current inventions provide manufacturing processes that are capable of robustly producing high-quality protein-based therapeutics at 2 kL and 10 kL scale. Overall, SPTFF provides the tools to address many volume-related facility-fit constraints associated with high-titer processes.

The invention provides method for purifying high bioreactor titer from less than 1 g/L to high-titer of more than 20 g/L. The high-titer processes provide increased manufacturing plant productivity, allowing to meet the growing demand for therapeutic medicines.

### Cell Culture

The present inventions are amenable for production of therapeutic proteins in cell culture, such as mammalian cell culture. Exemplary cell lines are CHO, Per.C6 cells, Sp2/0 cells, HeLa and HEK293 cells. CHO cells include, but are not limited to, CHO-ori, CHO-K1, CHO-s, CHO-DHB11, CHO-DXB11, CHO-K1 SV, and mutants and variants thereof. HEK293 cells include, but are not limited, to HEK293, HEK293A, HEK293E, HEK293F, HEK293FT, HEK293FTM, HEK293H, HEK293MSR, HEK293S, HEK293SG, HEK293SGGD, HEK293T and mutants and variants thereof. Other suitable cells include, but are not limited to BHK (baby hamster kidney) cells, HeLa cells and Human Amniotic cells, such as Human Amniotic Epithelial cells. Other cell types for production include insect cells, such as Sf9.

### Buffers

The buffers used in the methods of purifying a therapeutic protein, according to the inventions, can comprise at least one buffering agent, including but not limited to Bis-Tris-Propane (BTP), Bis-Tris, Tris, Glycine, Bicine, Tricine, Acetate, Borate, Citrate, Carbonate, Phosphate, Formate, Sulfate, Succinic acid, Sulfonic acid and variants thereof (for example MES, PIPES, HEPES, CHES, CAPS, MMS, PBMS), Diethanolamine, and Imidazole.

The buffers used in the methods of purifying a therapeutic protein, according to the inventions, can comprise about 1 mM to about 50 mM, about 1 mM to about 5 mM, about 5 mM to about 10 mM, about 10 mM to about 15 mM, about 15 mM to about 20 mM, about 20 mM to about 25 mM, about 25 mM to about 30 mM, about 30 mM to about 35 mM, about 35 mM to about 40 mM, about 40 mM to about 45 mM, or about 45 mM to about 50 mM buffering agent. The strength of the buffering agent can be about 5 mM±1 mM, about 10 mM±1 mM, about 15 mM±1 mM, about 20 mM±1 mM, about 25 mM±1 mM, about 30 mM±1 mM, about 35 mM±1 mM, about 40 mM±1 mM, about 45 mM±1 mM, or about 50 mM±1 mM. The process can comprise about 5 mM, about 10 mM, about 15 mM, about 20 mM, about 25 mM, about 30 mM, about 35 mM, about 40 mM, about 45 mM, or about 50 mM buffering agent.

### Salts

The salt in the buffers used in the methods of purifying a therapeutic protein, according to the inventions, can be any organic or inorganic salt, include, but are not limited to, metal salts such as sodium, potassium and cesium salts; alkaline earth metal salts such as calcium and magnesium salts; organic amine salts such as triethylamine, guanidine and N-substituted guanidine salts, acetamidine and N-substituted acetamidine, pyridine, picoline, ethanolamine, triethanolamine, dicyclohexylamine, and N,N′-dibenzylethylenediamine salts. Suitable salts (of basic nitrogen centers) include, but are not limited to, inorganic acid salts such as the hydrochloride, hydrobromide, sulfate, phosphate; organic acid salts such as trifluoroacetate and maleate salts; sulfonates such as methanesulfonate, ethanesulfonate, benzenesulfonate, p-toluenesulfonate, camphor sulfonate and naphthalenesulfonate; amino acid salts such as arginate, alaninate, asparginate and glutamate; and carbohydrate salts such as gluconate and galacturonate. Non-limiting examples of acceptable salts include, without limitation, sodium salts, ammonium salts, potassium salts, calcium salts, and magnesium salts (e.g., sodium, ammonium, potassium, calcium, and magnesium chloride; sodium, ammonium, potassium, calcium and magnesium acetate; sodium, ammonium, potassium, calcium and magnesium citrate; sodium, ammonium, potassium, calcium and magnesium phosphate; sodium, ammonium, potassium, calcium and magnesium fluoride; sodium, ammonium, potassium, calcium and magnesium bromide; and sodium, ammonium, potassium, calcium and magnesium iodide). The acceptable salt can be sodium chloride or arginine hydrochloride (L-arginine hydrochloride).

The concentration of the salt in the buffer can be about 50 mM to about 500 mM, about 50 mM to about 400 mM, about 50 mM to about 350 mM, about 50 mM to about 300 mM, about 50 mM to about 250 mM, about 50 mM to about 200 mM, about 50 mM to about 150 mM, about 50 mM to about 100 mM, about 50 mM, about 100 mM, about 150 mM, about 200 mM, about 250 mM, about 300 mM, about 350 mM, about 400 mM, about 450 mM, or about 500 mM.

The concentration of the salt in the buffer can be about 100±10 mM, about 150±10 mM, about 200±10 mM, about 250±10 mM, about 300±10 mM, about 350±10 mM, about 400±10 mM, about 450±10 mM, about 100±20 mM, about 150±20 mM, about 200±20 mM, about 250±20 mM, about 300±20 mM, about 350±20 mM, about 400±20 mM, or about 450±20 mM.

pH

The pH of the buffers used in the methods of purifying a therapeutic protein, according to the inventions, can be about 3.5 to about 11.0, about 3.5 to about 10.5, about 3.5 to about 10.0, about 3.5 to about 9.5, about 3.5 to about 9.0, about 3.5 to about 8.5, about 3.5 to about 8.0, about 3.5. to about 7.5, about 3.5 to about 7.0, about 3.5 to about 6.5, about 3.5 to about 6.0, about 3.5 to about 5.5, about 3.5 to about 5.0, about 3.5 to about 4.5, about 3.5 to about 4.0, about 4.5 to about 10.5, about 4.5 to about 10.0, about 4.5 to about 9.5, about 4.5 to about 9.0, about 4.5 to about 8.5, about 4.5 to about 8.0, about 4.5. to about 7.5, about 4.5 to about 7.0, about 4.5 to about 6.5, about 4.5 to about 6.0, about 4.5 to about 5.5, about 4.5 to about 5.0, about 5.5 to about 10.5, about 5.5 to about 10.0, about 5.5 to about 9.5, about 5.5 to about 9.0, about 5.5 to about 8.5, about 5.5 to about 8.0, about 5.5. to about 7.5, about 5.5 to about 7.0, about 5.5 to about 6.5, about 5.5 to about 6.0, about 6.5 to about 10.5, about 6.5 to about 10.0, about 6.5 to about 9.5, about 6.5 to about 9.0, about 6.5 to about 8.5, about 6.5 to about 8.0, about 6.5. to about 7.5, about 6.5 to about 7.0, about 7.5 to about 10.5, about 7.5 to about 10.0, about 7.5 to about 9.5, about 7.5 to about 9.0, about 7.5 to about 8.5, about 7.5 to about 8.0, about 8.5 to about 10.5, about 8.5 to about 10.0, about 8.5 to about 9.5, about 8.5 to about 9.0, about 9.0 to about 10.5, about 9.0 to about 10.0, about 9.0 to about 9.5, about 9.5 to about 10.5, about 9.5 to about 10.0, about 10.0 to about 11.0, about 10.0 to about 10.5, or about 10.5 to about 11.0. The pH of the buffer can be about 3.5, about 4.0, about 4.5, about 5.0, about 5.5, about 6.0, about 6.5, about 7.0, about 7.5, about 8.0, about 8.5, about 9.0, about 9.5, about 10.0, about 10.5, or about 11.0. The pH of the buffer can be about 4.0±0.05, about 4.5±0.05, about 5.0±0.05, about 5.5±0.05, about 6.0±0.05, about 6.5±0.05, about 7.0±0.1, about 7.0±0.05, about 7.5±0.1, about 7.5±0.05, about 8.0±0.1, about 8.0±0.05, about 8.5±0.1, about 8.5±0.05, about 9.0±0.1, about 9.0±0.05, about 9.5±0.1, about 9.5±0.05, about 10.0±0.1, about 10.0±0.05, about 10.5±0.1, about 10.5±0.05 about 11.0±0.1, or about 11.0±0.05.

### Surfactants

The buffers used in the methods of purifying a therapeutic protein, according to the inventions, can comprises from about 0.001% (w/v) to about 0.3% (w/v) non-ionic surfactant, such as poloxamers (e.g., poloxamer 188 (P188)). The buffer can comprise about 0.001% (w/v), about 0.0015% (w/v), about 0.002% (w/v), about 0.0025% (w/v), about 0.003% (w/v), about 0.0035% (w/v), about 0.004% (w/v), about 0.0045% (w/v), about 0.005% (w/v), about 0.0055% (w/v), about 0.006% (w/v), about 0.0065% (w/v), about 0.007% (w/v), about 0.0075% (w/v), about 0.008% (w/v), about 0.0085% (w/v), about 0.009% (w/v), or about 0.0095% (w/v) non-ionic surfactant P188. The buffer can comprise about 0.01% (w/v), about 0.015% (w/v), about 0.02% (w/v), about 0.025% (w/v), about 0.03% (w/v), about 0.035% (w/v), about 0.04% (w/v), about 0.045% (w/v), about 0.05% (w/v), about 0.055% (w/v), about 0.06% (w/v), about 0.065% (w/v), about 0.07% (w/v), about 0.075% (w/v), about 0.08% (w/v), about 0.085% (w/v), about 0.09% (w/v), about 0.095% (w/v), about 0.10% (w/v), about 0.15% (w/v), about 0.20% (w/v), about 0.25% (w/v), or about 0.30% (w/v) non-ionic surfactant P188. The buffer can comprise 0.20% w/v±0.01% w/v non-ionic surfactant P188. The buffer of the present disclosure can comprises about 0.001% (w/v), about 0.0015% (w/v), about 0.002% (w/v), about 0.0025% (w/v), about 0.003% (w/v), about 0.0035% (w/v), about 0.004% (w/v), about 0.0045% (w/v), about 0.005% (w/v), about 0.0055% (w/v), about 0.006% (w/v), about 0.0065% (w/v), about 0.007% (w/v), about 0.0075% (w/v), about 0.008% (w/v), about 0.0085% (w/v), about 0.009% (w/v), about 0.0095% (w/v), or about 0.01% (w/v) poloxamer 188. The buffers of the present disclosure comprises about 0.005% w/v±0.001% w/v polysorbate 80 or about 0.005% w/v±0.001% w/v poloxamer 188.

### Viral Inactivation Pool Filtration (VIPF)

The following protocol is exemplary and not limiting. VIPF can be performed using, for example, required numbers of Millistak+® HC Pro Depth Filter XOSP filters are connected in parallel in a filter housing. The filter is flushed with RODI using pump and then flushed with an appropriate process buffer. Air blowdown can be performed at 10 psi inlet pressure. The feed can be filtered at Less than or equal to 300 LMH (liter/m²/hour) through the Millistak+® HC Pro Depth Filter XOSP filters and pressure can be monitored at the filter inlets and outlets. Post filtration, a buffer flush of 10 L/m² can be performed. Other filters include, but are not limited to, Emphaze and Lifeassure.

The VIPF pool can have a back pressure of about 3 psi to about 15 psi, about 3 psi, about 4 psi, about 5 psi, about 6 psi, about 7 psi, about 8 psi, about 9 psi, about 10 psi, about 11 psi, about 12 psi, about 13 psi, about 14 psi, about 15 psi, about 6±0.5 psi, about 8±0.5 psi, about 10±0.5 psi, about 12±0.5 psi, or about 14±0.5 psi.

The inventions advantageously provide methods of purifying therapeutic proteins from cell cultures of high-titer bioreactor harvest pool, wherein the method can comprise filtering viral inactivation (VI) pool through a VI pool filtration (VIPF) system serially connected to a single pass tangential flow filtration (SPTFF) system to in-line concentrate and reduce the pool volume for subsequent purification steps. According to the inventions, the high-titer cell culture protein concentration in the bioreactor can be from at least 5 g/L to about 50 g/L, about 6 g/L to about 50 g/L, about 7 g/L to about 50 g/L, about 8 g/L to about 50 g/L, about 9 g/L to about 50 g/L, about 10 g/L to about 50 g/L, about 11 g/L to about 50 g/L, about 12 g/L to about 50 g/L, about 13 g/L to about 50 g/L, about 14 g/L to about 50 g/L, about 15 g/L to about 50 g/L, about 16 g/L to about 50 g/L, about 17 g/L to about 50 g/L, about 18 g/L to about 50 g/L, about 19 g/L to about 50 g/L, about 20 g/L to about 50 g/L, about 21 g/L to about 50 g/L, about 22 g/L to about 50 g/L, about 23 g/L to about 50 g/L, about 24 g/L to about 50 g/L, about 25 g/L to about 50 g/L, about 26 g/L to about 50 g/L, about 27 g/L to about 50 g/L, about 28 g/L to about 50 g/L, about 29 g/L to about 50 g/L, about 30 g/L to about 50 g/L, about 31 g/L to about 50 g/L, about 32 g/L to about 50 g/L, about 33 g/L to about 50 g/L, about 34 g/L to about 50 g/L, about 35 g/L to about 50 g/L, about 36 g/L to about 50 g/L, about 37 g/L to about 50 g/L, about 38 g/L to about 50 g/L, about 39 g/L to about 50 g/L, about 40 g/L to about 50 g/L, about 41 g/L to about 50 g/L, about 42 g/L to about 50 g/L, about 43 g/L to about 50 g/L, about 44 g/L to about 50 g/L, about 45 g/L to about 50 g/L, about 46 g/L to about 50 g/L, about 47 g/L to about 50 g/L, about 48 g/L to about 50 g/L, about 49 g/L to about 50 g/L. The high-titer cell culture protein concentration in the bioreactor can be about 6.0±0.5 g/L, about 7.0±0.5 g/L, about 8.0±0.5 g/L, about 9.0±0.5 g/L, about 10.0±0.5 g/L, about 11.0±0.5 g/L, about 12.0±0.5 g/L, about 13.0±0.5 g/L, about 14.0±0.5 g/L, about 15.0±0.5 g/L, about 16.0±0.5 g/L, about 17.0±0.5 g/L, about 18.0±0.5 g/L, about 19.0±0.5 g/L, about 20.0±0.5 g/L, about 21.0±0.5 g/L, about 22.0±0.5 g/L, about 23.0±0.5 g/L, about 24.0±0.5 g/L, about 25.0±0.5 g/L, about 26.0±0.5 g/L, about 28.0±0.5 g/L, about 30.0±0.5 g/L, about 32.0±0.5 g/L, about 34.0±0.5 g/L, about 36.0±0.5 g/L, about 38.0±0.5 g/L, about 40.0±0.5 g/L, about 42.0±0.5 g/L, about 44.0±0.5 g/L, about 46.0±0.5 g/L, about 48.0±0.5 g/L, or about 49.0±0.5 g/L.

The SPTFF system of the inventions can comprises at least 1 to 20 SPTFF capsules, 1 to 2 SPTFF capsules, 2 to 3 SPTFF capsules, 3 to 4 SPTFF capsules, 4 to 5 SPTFF capsules, 5 to 6 SPTFF capsules, 6 to 7 SPTFF capsules, 7 to 8 SPTFF capsules, 8 to 9 SPTFF capsules, 9 to 10 SPTFF capsules, 10 to 11 SPTFF capsules, 11 to 12 SPTFF capsules, 12 to 13 SPTFF capsules, 13 to 14 SPTFF capsules, 14 to 15 SPTFF capsules, 15 to 16 SPTFF capsules, 16 to 17 SPTFF capsules, 17 to 18 SPTFF capsules, 18 to 19 SPTFF capsules, or 19 to 20 SPTFF capsules.

The SPTFF capsules, according to the inventions, can be connected in a serial, parallel or a combination of serial and parallel configuration.

The concentrating and reducing the VI pool volume through VIPF-SPTFF concentrates and reduces the pool volume to about 10% to 90%, 15% to 85%, 20% to 80%, 25% to 75%, 30% to 70%, 35% to 65%, 40% to 60%, 45% to 55%, 20% to 70%, 20% to 60%, 20% to 50%, 30% to 80%, 30% to 70%, 30% to 60%, 30% to 50%, 30% to 40%, 30% to 80%, 30% to 60%, 30% to 50%, 30% to 40%, 40% to 80%, 40% to 70%, 40% to 50%, 50% to 80%, 50% to 70%, 60% to 80%, 60% to 70%, 70% to 90%, 70% to 80%, 80% to 85%, 80% to 90%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 70% to 75%, about 80% to 85%, about 85% to 90%, about 10%, about 15%, about 20%, about 25%, about 30%, about 35%, about 40%, about 45%, about 50%, about 55%, about 60%, about 65%, about 70%, about 75%, about 80%, about 85%, about 90%, about 20±1%, about 30±1%, about 35±1%, about 40±1%, about 43±1%, about 46±1%, about 49±1%, about 51±1%, about 53±1%, about 56±1%, about 59±1%, or about 61±1% of the VI pool volume.

### EXAMPLES

The inventions are further described by the following Examples, which do not limit the inventions in any manner and are applicable to all sections of the descriptions of the inventions and the aspects of the inventions. The order of performance of the below Examples can be altered or combined as determined by the person of skill in the art in view of the teachings and data contained herein.

### Example 1

### Plant Fit Volume

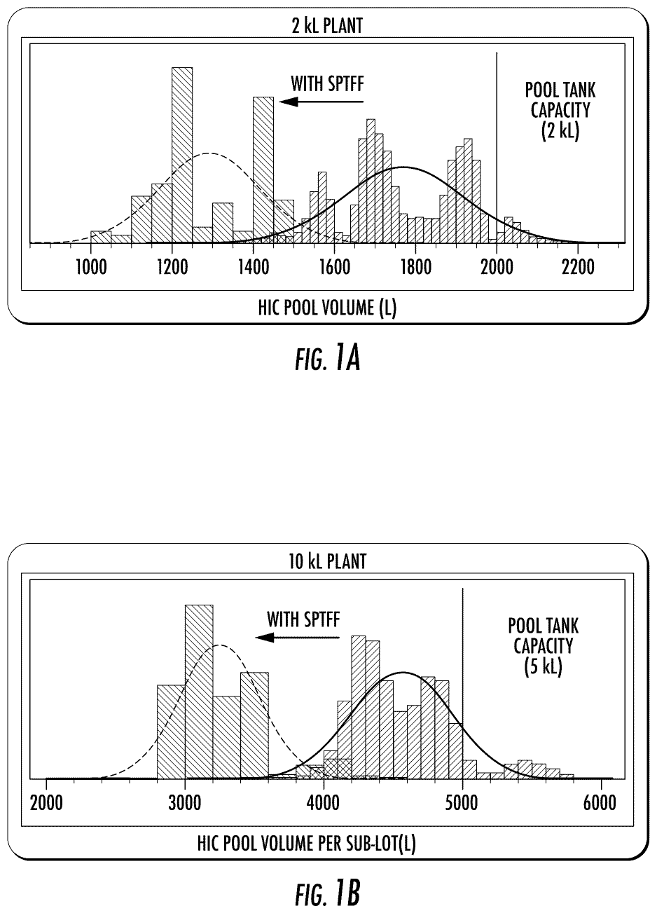

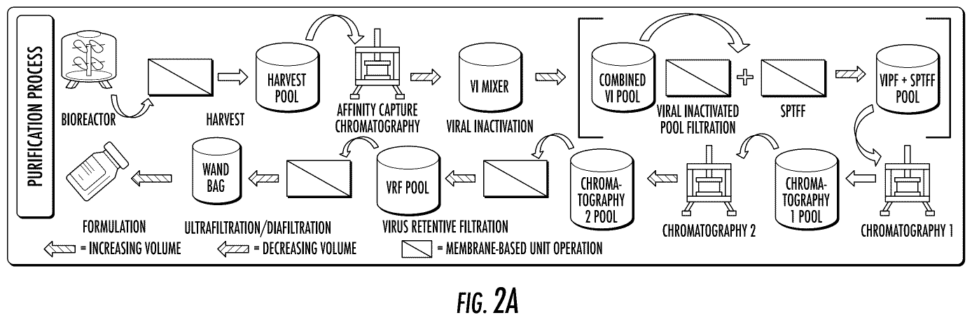

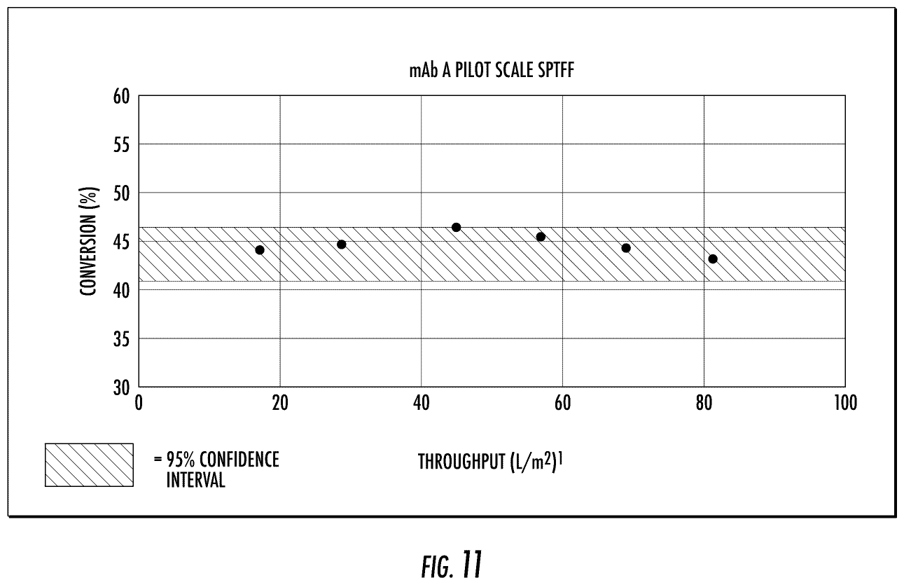

FIGS. 1A and 1B are graphs concerning production plants containing 2,000 L (FIG. 1A) and 10,000 L (FIG. 1B) bioreactors showing the pool volumes following the hydrophobic interaction chromatography (HIC) step for a mAb with bioreactor titer of 12 g/L with SPTFF and without SPTFF. Plant fit shows intermediate pool volumes at the manufacturing scale using molecule specific bioreactor productivity and yield of various unit operations. The HIC unit operation is often the first point in the purification process that plant fit failures are observed due to exceeding the volume constraint of the pool tank.

The bioreactor titer of 12 g/L that was implemented with SPTFF purification process with a 0.74 standard deviation for bioreactor titer. SPTFF conversion was applied as a constant to filtered viral inactivated pool volumes (e.g., for 50% conversion, all viral inactivated pool filtration (VIPF) pool was divided by 2). The pool volume distributions are generated, with about a 50% reduction in the viral inactivated pool filtration (VIPF) volume when SPTFF is incorporated into the purification process, compared to a process with no SPTFF. No instances of exceeding fill volume of pool tank are observed for various intermediate purification pools for a mAb process with high-titer cell culture having protein concentration in the bioreactor of about 12 g/L.

Conversion calculation equations are as shown below:

$$Conversion = \frac{permeate flow rate}{feed flow rate} \times 100 \qquad Equation 1 : Conversion$$

$$Concentration Factor = \frac{retentate protein concentration}{feed protein concentration} \qquad Equation 2 : Concentration Factor$$

Conversion is the percent of liquid removed from the feed. 50% conversion is equivalent to a 2× concentration.

### Example 2

### Purification Process

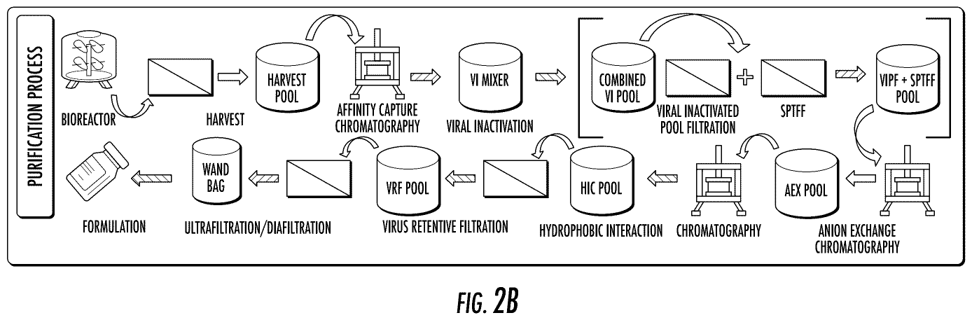

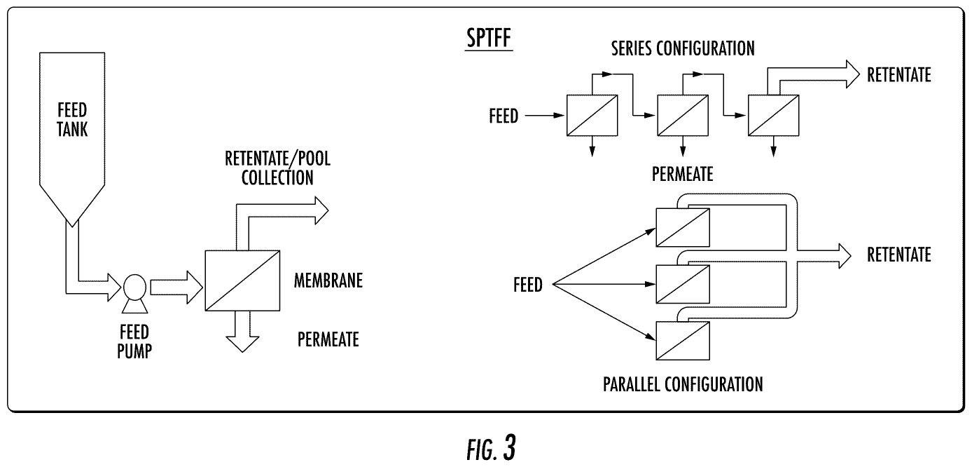

FIGS. 2A and 2B schematically depicts exemplary high-titer purification processes incorporating SPTFF. FIG. 2A depicts pools following chromatography 1 and chromatography 2. Chromatography 1 can be an AEX, CEX or an HIC column, and chromatography 2 can be an be an AEX, CEX or an HIC column, and chromatography 1 and 2 can be the same or different, preferably different.

FIG. 2B depicts AEX and HIC chromatographs following SPTFF. As depicted in the downstream of FIGS. 2A and 2B, the viral inactivated pool filtration (VIPF) step is determined to be more advantageous compared with SPTFF capsules attached to the outlet of the anion exchange chromatography (AEX) unit. Advantages of incorporating SPTFF include increased flexibility regarding the impact of minimum operating flow rate on impurity removal during VIPF. Back pressure on the VIPF filter also is lower risk than back pressure on an AEX column. A consistent VIPF outlet stream compared to cyclical polishing pool streams additionally provides a more straightforward SPTFF operation requiring less alterations throughout the run. This comparison also outlined the need for analysis of impurity removal of the paired VIPF+SPTFF step (FIGS. 2A and 2B), the impact of feed protein concentration on SPTFF, and increased concentration feeds for further purification steps. In some aspects, the order and type of chromatography column and filtration can be altered according to teachings contained herein.

SPTFF is incorporated at downstream in-line of normal flow filtration process such as VIPF. The advantages regarding where in the purification process to incorporate the SPTFF include:
Reduced pressure concern of SPTFF on VIPF;
Consistent SPTFF feed protein concentration and
Increased VIPF process flow rate flexibility.

### Example 3

### SPTFF Configurations

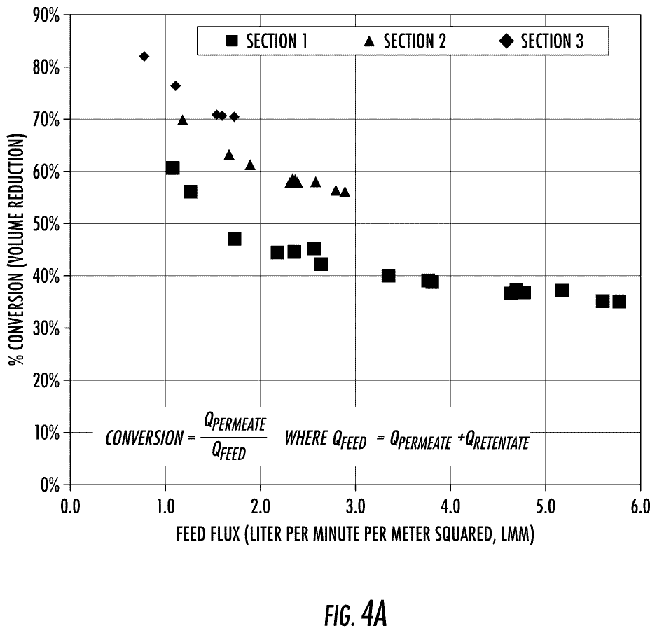

SPTFF allows for an increase in the feed protein concentration, for example, to a desired level, in a single pass across semi-permeable membranes. The SPTFF setup is simple and modular, which allows connection to various unit operations in the purification process. The specific membrane configuration can be modified allowing for process optimization. FIG. 3 depicts Millipore Sigma Pellicon® SPTFF capsules filter configurations (Series configuration and Parallel configuration) for SPTFF.

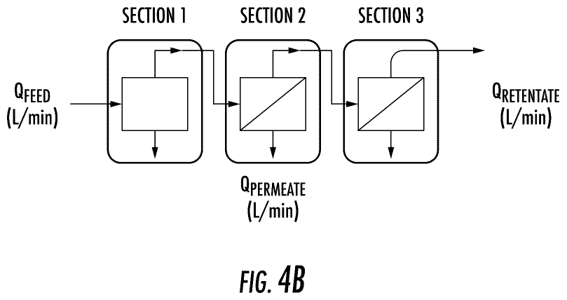

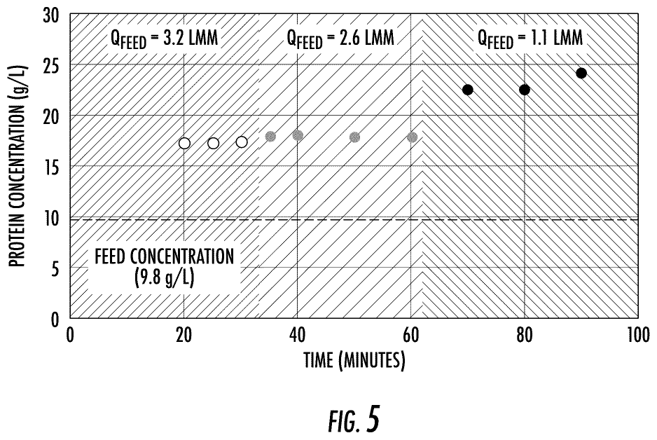

SPTFF experiments initially included an optimization testing of capsules with, for example, a desired conversion level. To determine the number of capsules needed for a desired conversion level, feed flux excursions at various back pressures were performed with Millipore Sigma onsite. The results suggested 1 or 2 sections in series can achieve a desired conversion level with a stable back pressure of about 8 psi (FIG. 4A).

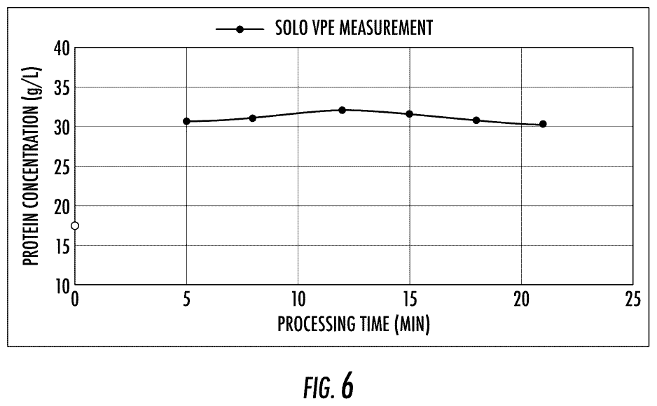

FIGS. 4A and 4B shows Feed flux excursion experiments (FIG. 4A) performed using the Millipore Sigma 0.1 m² Pellicon® SPTFF capsules (experiment set-up illustrated in FIG. 4B) showed that 2 sections in series configuration are optimal. All sections are in connected a series configuration. ‘Section 3’ refers to a set-up with 3×0.1 m² capsules in a series configuration. Flux is calculated using the cumulative area of all sections in each set-up. FIG. 5 shows the results of experiments that demonstrate for a given feed concentration, the SPTFF pool protein concentration (and the percentage conversion) can be varied by changing feed flux at a fixed back pressure of about 8 psi.

Several experiments testing various feed flux and feed protein concentrations were performed testing sections 1-3 in both series and parallel configurations (FIGS. 3 and 4B) to generate a dataset that was used to create the profiler. This profiler helped determine 2 sections in series configuration for subsequent experiments combined with VIPF.

The results of the experiments evaluating a range of feed flux and feed protein concentration values with varying numbers of 0.1 m² capsules connected in a series or a parallel configuration. These results are used for determining the feed flux set point for achieving, for example, a desired conversion level for a given feed concentration and for the fixed SPTFF set-up comprising 2 capsules attached in a series configuration.

Ranges studied in the profiler are as follows: Feed flux, 0.52 to 4.0 liters per minute per meters squared (LMM); Number of sections 1, 2, 3 (each section has membrane area of either 0.0088 m² 0.1 m² or 0.5 m²) Configuration, series or parallel; Feed concentration ranging from 7.6 to 31.2 g/L. These ranges detail the lowest and highest values assessed for each factor.

### Example 4

Pairing SPTFF with VIPF

A profiler was used to determine set points for a purification process implemented with the SPTFF directly in line downstream of an VIPF filter for analysis of impact to quality attributes, pressures, and conversion. The setup used a 0.78 m² LifeAssure PDA filter with 2×0.5 m² Millipore SPTFF capsules in a series configuration. Quality data analyzed for VIPF+SPTFF experiments showed no significant impact to host cell protein (HCP) or deoxyribonucleic acid (DNA) resulting from the addition of the SPTFF to the process. Quality attributes test results for VIPF+SPTFF and VIPF without SPTFF are summarized in Table 1.

TABLE 1

| | VIPF + SPTFF | | | VIPF without SPTFF | | |
|---|---|---|---|---|---|---|
| | HCP | DNA | HMW | HCP | DNA | HMW |
| | (ppm) | (ppb) | (%) | (ppm) | (ppb) | (%) |
| ProA Eluate (1 Day Hold) | 1660 | 0.365 | 10.12 | 1730 | NM | 9.58 |
| VIP | 1460 | 17.6 | 10.03 | 1180 | 0.32 | 9.85 |
| VIPF + SPTFF Pool | 1450 | 0.3005 | 10.38 | N/A | N/A | 10.4 |
| AEX Load | 1477 | 0.3425 | 10.4 | N/A | N/A | 12.3 |
| AEX Pool | 269 | 11.0 | 8.08 | 146 | <LOQ | 9.85 |

Advantages of Pairing SPTFF with VIPF include:
VIPF can be operated with variable flow rates, offering SPTFF process flexibility;
Fixed flow rates of chromatography columns would require large amounts of SPTFF filter area to meet feed flux and a desired conversion level, or optionally an additional fixed pool tank; and
Pairing SPTFF with the VIPF unit operation provides the most operational flexibility.

Table 2. VIPF-SPTFF Verification with XOSP & Emphaze Filter using a mAb. The experiments were performed with 0.0023 m² Millistak+® XOSP or 0.0025 m² 3M Emphaze VIPF filters in line with 2×0.0088 m² Millipore Pellicon 3 Biomax in a series configuration with diverter plates.

TABLE 2

| | Experiment with | | Experiment with | |
|---|---|---|---|---|
| | Emphaze VIPF | | X0SP VIPF | |
| | | SPTFF | | SPTFF |
| VIP Filtration | VIPF | inline | VIPF | inline |
| Parameters | Only | VIPF | Only | VIPF |
| VIPF Filter area | 25 cm² | 25 cm² | 23 cm² | 23 cm² |
| SPTFF Filter area | — | 0.0176 cm² | — | 0.0176 cm² |
| Filter Loading | 155 | 200 | 100 | 100 |
| (L/m²) | | | | |
| VIP Filtration | 98.4 | 99.8 | 93.9 | 98.8 |
| Yield (%) | | | | |
| HMW dimer/HMW | 3.55/0.24 | 3.77/0.28 | 3.02/0.17 | 3.41/0.17 |
| higher order (%) | | | | |
| HCP (ppm) | 819 | 792 | 554 | 428 |
| VIPF Pressure | 1.1 | 0.9 | 2.4 | 1.50 |
| Drop (psi) | | | | |
| Conversion (%) | N/A | 43.1 | N/A | 47.2 |

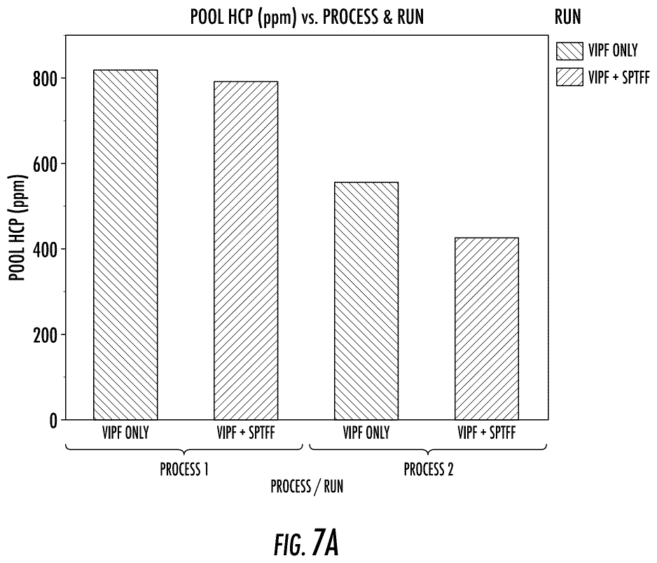

A test run performed with SPTFF setup with 2×0.11 m² Millistak+® XOSP VIPF filters in line with 2×0.5 m² Pellicon® SPTFF capsules in a series configuration to assess the scalability. Concentration reached a steady state protein concentration in approximately 5 minutes and output a consistent pool after that time point for the duration of the run at a conversion of approximately 43% (FIG. 6). Quality data are shown in Tables 1-5.

Impact of two-fold protein concentration feeds on downstream unit operations AEX and HIC also were evaluated. No impact to HCP, DNA, or high molecular weight (HMW) aggregates was observed because of the increased load concentration (see Tables 1-5).

TABLE 3
Pilot Scale and Bench scale VIPF +
SPTFF Comparison Runs using a mAb.

| VIP Filtration Parameters | Bench Scale | Pilot Scale |
|---|---|---|
| VIPF Filter area (m²) | 0.0046 | 0.22 |
| SPTFF Filter area (m²) | 0.0176 | 1.0 |
| VIPF:SPTFF Filter area ratio | 0.26 | 0.22 |
| VIPF Filter Loading (L/m²) | 100 | 99.3 |
| Filtration Yield (%) | 87.0^{a} | 92.2^{b} |
| HMW Dimer/Higher Order (%) | 4.19/0.19 | 4.36/0.22 |
| HCP (ppm) | 369 | 438 |
| VIPF Pressure Drop (psi) | 1.5 | 3.1 |
| Conversion (%) | 48.2 | 41.7 |

^{a}Issues with the pump impacted the recovery from the lines.
^{b}2% of 100% total lost to the venting of the X0SP filters.

### Example 5

### Process Performance and Product Qualities

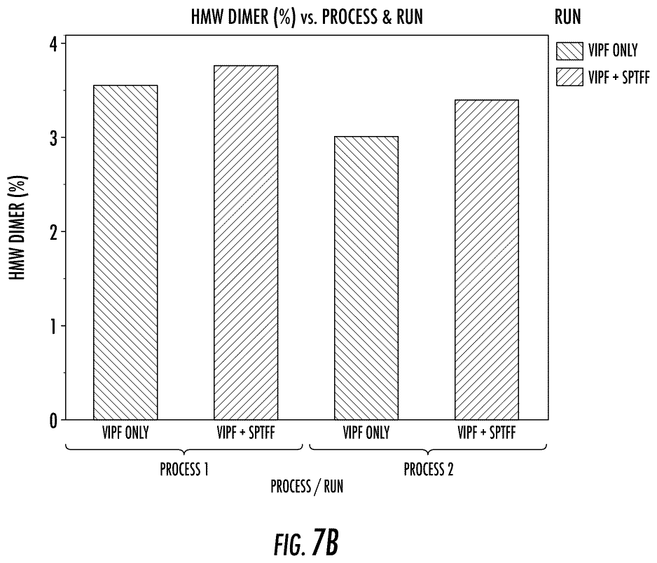

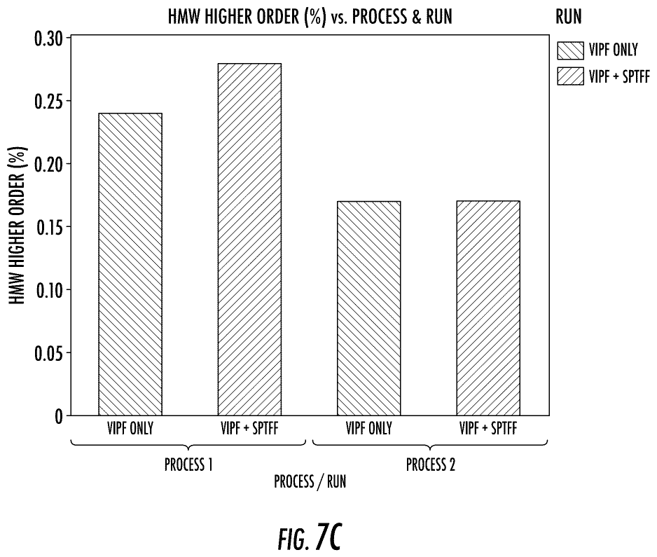

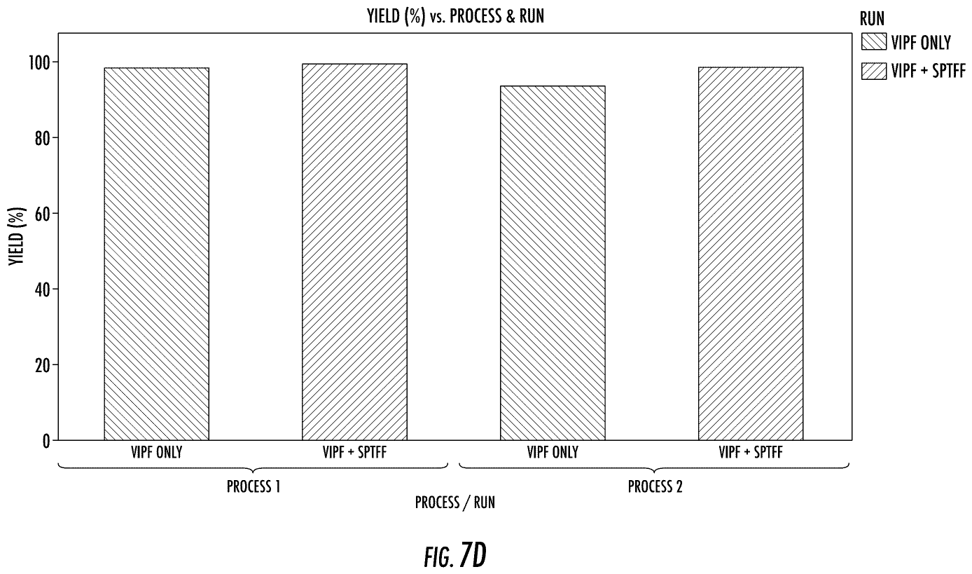

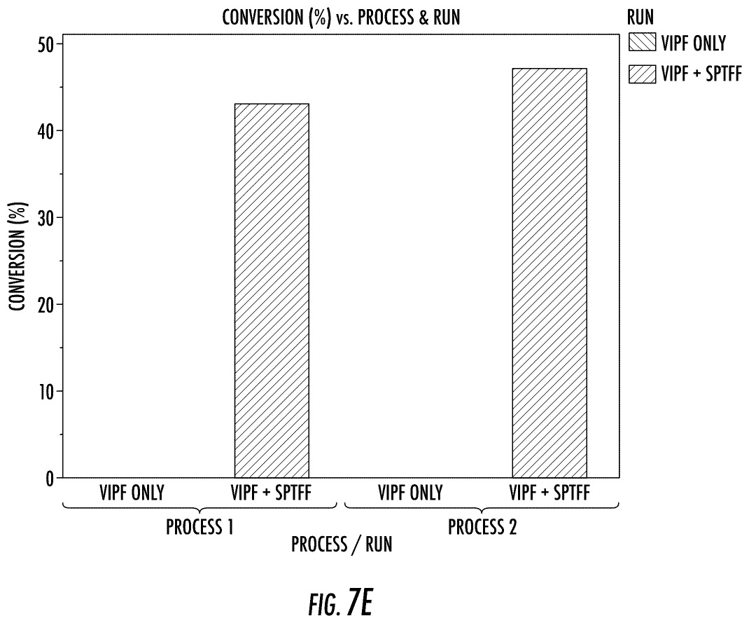

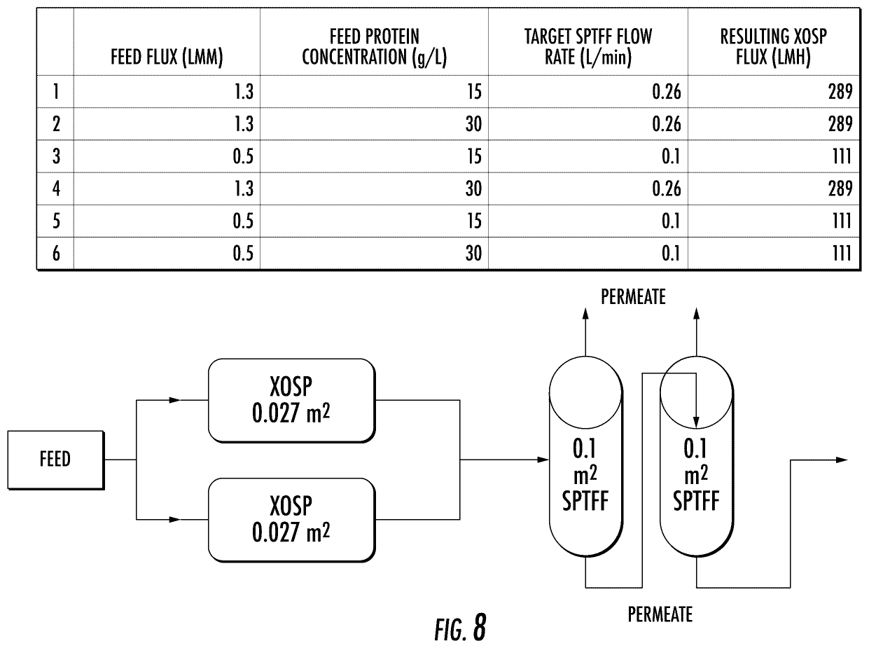

FIGS. 7A to 7E showing results of the VIPF alone and VIPF+SPTFF process performance and product qualities for the two VIPF platform processes. Conversions of about 43.1% and 47.2% were observed for process 1 and process 2, respectively. Process 1 refers to subjecting the VI pool to VI pool filtration (VIPF) system (Emphaze) with or without SPTFF. Process 2 refers to subjecting the VI pool to VI pool filtration (VIPF) system (XOsp) with or without SPTFF. Both processes relate to concentrating the pool volume for generating the VIPF-SPTFF pool.

VIPF+SPTFF showed: (1) lower host cell proteins than VIPF-only (FIG. 7A), (2) an increase in the high molecular weight dimer % than VIPF alone (HMW Dimer %) (FIG. 7B), (3) HMW higher order (%) for process 1 and nearly equal for process 2 than VIPF alone (FIG. 7C), (4) a higher yield percentage than VIPF alone (FIG. 7D), and (5) a significantly higher conversion (%) than VIPF alone (FIG. 7E). The data shown are for a monoclonal antibody.

TABLE 4
AEX High Concentration Small Scale Runs using a mAb.

| AEX Variables | Unconcentrated | | Medium | | High Concentrated | | |
|---|---|---|---|---|---|---|---|
| and Parameters | Feed | | Concentrated Feed | | Feed | | |
| Feed | 13.3 | | 31.1 | | 57.9 | | |
| Concentration | | | | | | | |
| (g/L) | | | | | | | |
| Load HMW (%) | 4.67 | | 4.85 | | 4.99 | | |
| Load HCP (ppm) | 43.9 | | 47.7 | | 48.8 | | |
| Resin | QSFF | POROS | QSFF | POROS | QSFF | | POROS |
| Column | 100 | 200 | 100 | 200 | 100 | 200 | 200 |
| Loading | | | | | | | |
| Pool HMW (%) | 2.80 | 3.99 | 2.61 | 4.15 | 2.68 | 3.48 | 4.32 |
| Pool HCP (ppm) | 19.9 | 28 | 12.9 | 24 | 17.8 | 24.2 | 23 |
| Yield (%) | 92.9 | 98.3 | 91.2 | 96.6 | 91.3 | 95.6 | 99.0 |
| Concentration | 8.04 | 10.2 | 12.3 | 18.4 | 15.3 | 24.8 | 25.6 |
| (g/L) | | | | | | | |

The SPTFF process, utilizing Millipore Sigma capsules, combined with the VIPF unit operation has been shown to be effective, minimally impactful to quality attributes, and scalable. The current condition tested to be the most effective at achieving an exemplary conversion level, here about 8 psi system back pressure with 1 or 2×SPTFF capsule sections in a series configuration, and an SPTFF flow rate of 0.2-3.0 LMM. The starting protein concentration of the step can alter the optimal condition, implying that each manufacturing process using SPTFF could require certain conditions to meet a desired concentration level. Based on these results, SPTFF can be implemented in line with intermediate process filtration, such as virus inactivated pool filtration (VIPF). The factors that often require optimization are the feed flow rate, membrane area in each section, and number of membrane sections which can be confirmed during the process development. The combined process can, therefore, be controlled by feed flow rate, back pressure, and also through in-line protein concentration monitoring using Raman Spectroscopy.

TABLE 5
HIC High Concentration Small Scale Runs using a mAb.

| | | | Medium | | High | |
|---|---|---|---|---|---|---|
| HIC Variables | Unconcentrated | | Concentrated | | Concentrated | |
| and Parameters | Feed | | Feed | | Feed | |
| Feed | 6.61 | | 10.8 | | 29.5 | |
| Concentration | | | | | | |
| (g/L) | | | | | | |
| Load HMW (%) | 1.81 | | 1.88 | | 1.97 | |
| Load HCP (ppm) | 35.0 | | 18.5 | | 15.3 | |
| Column Loading | 120 | 60 | 120 | 60 | 120 | 60 |
| (g/L) | | | | | | |
| Pool HMW (%) | 0.96 | 0.70 | 1.00 | 0.69 | 1.20 | 0.87 |
| Pool HCP (ppm) | 17.4 | 16.6 | 14.1 | 12.4 | 11.4 | 11.5 |
| Yield (%) | 96.0 | 92.1 | 95.1 | 91.4 | 94.9 | 93.2 |
| Pool | 5.33 | 4.43 | 7.50 | 5.74 | 14.3 | 9.34 |
| Concentration | | | | | | |

### Example 6

### Multivariate Experiments for Determination of the Key Quality Attributes

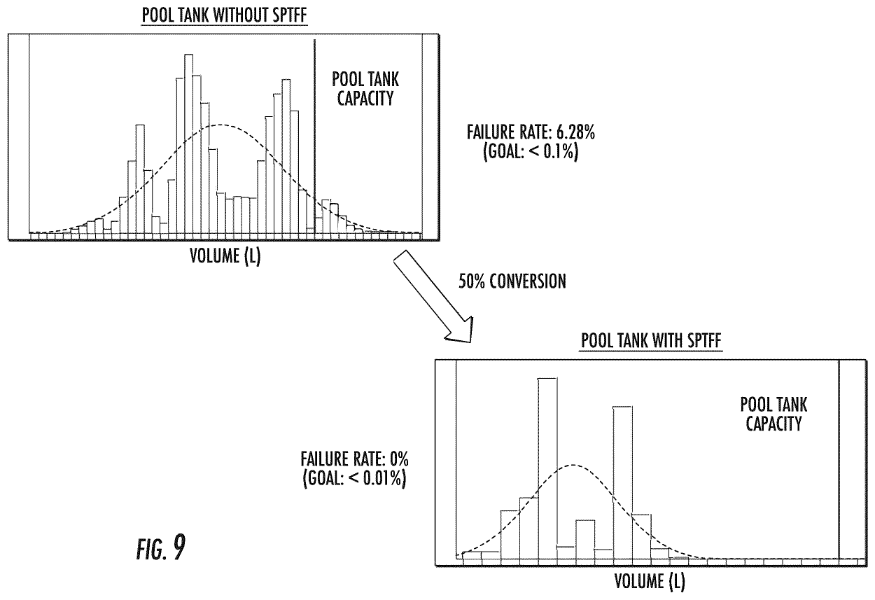

FIG. 8 shows a chart of multivariate experiments and schematic diagram for determination of the key quality attributes when the SPTFF process is combined with the VIPF unit operation using Millipore Sigma XOSP filters. In terms of feed flux (for the SPTFF capsules) 0.52 to 4.0 LMM and feed concentration ranging from 7.6 to 31.2 g/L can be tested. Results of the experiments can be used to identify acceptable parameter ranges for back pressure, feed flux, and membrane area to ensure success at manufacturing for a given program.

### Example 7

### Modeling

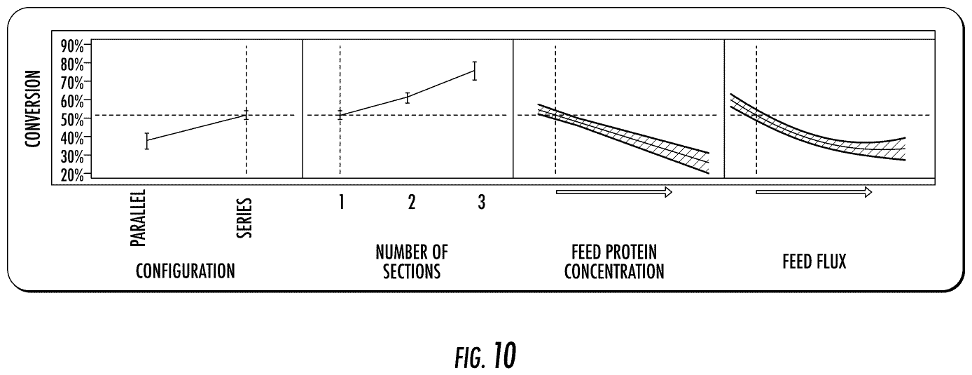

Using plant-fit models expected manufacturing loadings, and operational parameters, the estimated plant-fit pool volumes indicated a failure to fit in pool tanks following the hydrophobic interaction chromatography step for titers greater than 10 g/L. From this volume constraint, a volume reduction goal of 50% was determined to reduce predicted failures to 0% of 1 million simulated batches. Initial experimentation evaluating 1, 2, and 3 SPTFF capsules in series at a back pressure of 8 psi indicated that 1 or 2 capsules in series could achieve the 50% conversion goal. See FIG. 9.

Six experiments were performed with either parallel or series configurations of capsules and either 1, 2, or 3 sections in series. Operational parameters of feed protein concentration and feed flux ranged 7.6-31.2 g/L and 0.52-4.0 LMM, respectively. The conversion result from these six experiments was compiled using a statistical modeling software (JMP Statistical Discovery) and used to create a predictive model that can be used for informing process development. Results suggested series configuration, increasing number of capsules/sections, decreasing feed concentration, and decreasing flux would increase conversion. If a configuration and number of sections is selected, then based on the expected program feed concentration we could target a flux to achieve a target conversion. Alternatively, if a selected flux range is needed, more area could be used to increase conversion. This tool can be used to determine process conditions to transfer for GMP manufacturing & future scale up SPTFF applications. FIG. 10 schematically depicts a predictive model to inform process setpoints using program-specific inputs. FIG. 10 depicts percent conversion for configuration, number of sections, feed protein concentration and feed flux.

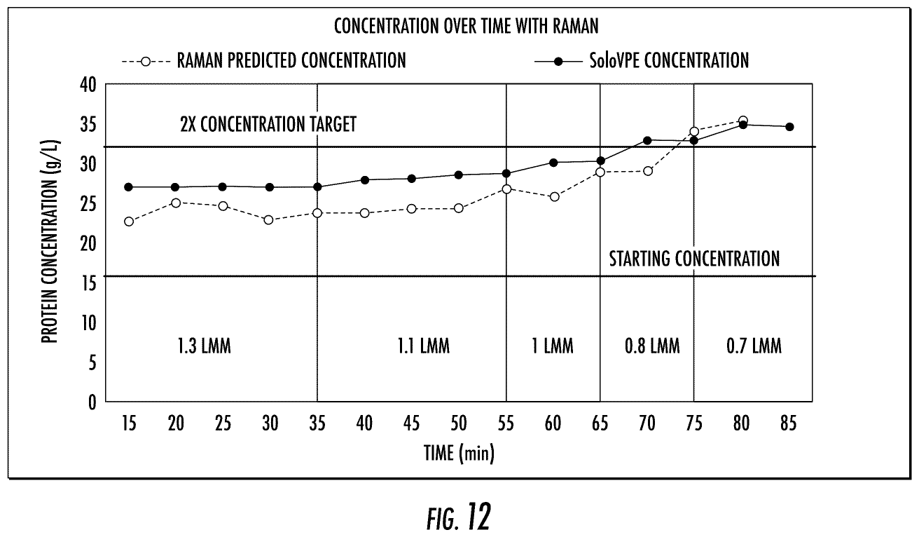

Using the small-scale model setpoints, a 5× scale-up run was conducted to assess conversion up to a VIPF filter throughput of ~80 L/m². Results showed that the small-scale model predicted range included the actual scale up conversion value. Conversion was within the model predicted 95% confidence interval for the entirety of the run. See FIG. 11.

### Example 8

### Experiment Procedure for Capsule Runs

Table 6 summarizes the general procedures followed for the experiments disclosed herein.

TABLE 6

| Step | Description | |
|---|---|---|
| Set-Up | 1. | Verify pump flow rates and make pump curve. |
| | 2. | Use flow rate sensors at bench scale (Leviflow sensor |
| | | by Levitronix). |
| | 3. | Connect pump, capsules, and pressure gauge or |
| | | Pendotech pressure sensors for pressure monitoring. |
| | 4. | Use ring stands or Millipore holder to support |
| | | capsules. |
| | 5. | Add lines to all the permeates and to the last |
| | | retentate. Connect the retentate of each capsule |
| | | to the feed of the subsequent capsule. |
| Flushing | 1. | New capsule: Flush capsules with RODI or |
| & Equil- | | equilibration buffer for ≥25. L/m² |
| ibration | 1. | Reused capsule: Sanitize capsules by recirculating |
| | | 0.5N NaOH for at least 30 minutes. Neutralize |
| | | system using RODI and ensure all lines are |
| | | neutral using pH paper. Prime system by |
| | | passing ≥25 L/m² of buffer matching |
| | | the components of the feed. |
| Experiment | 1. | Once system is primed, put feed line into feed |
| Procedure | | vessel and start pump. |
| | 2. | Adjust retentate valve to achieve, for example, |
| | | a target back pressure. |
| | 3. | If pre-use equilibration period was implemented: |
| | | Run system for at least 5 minutes for stabilization, |
| | | all lines should be placed into an “equilibrium |
| | | waste” bottle. |
| | 4. | Record flow rates of the pump, permeates, and |
| | | retentate based on volume per unit time throughput |
| | | run. |
| | 5. | If recycle mode is used: after all the material |
| | | passes through the pump, the pool and permeates are |
| | | recombined, mixed, and directed to the load line. |

### Example 9

SPTFF with Raman as a Tool for Monitoring and Control

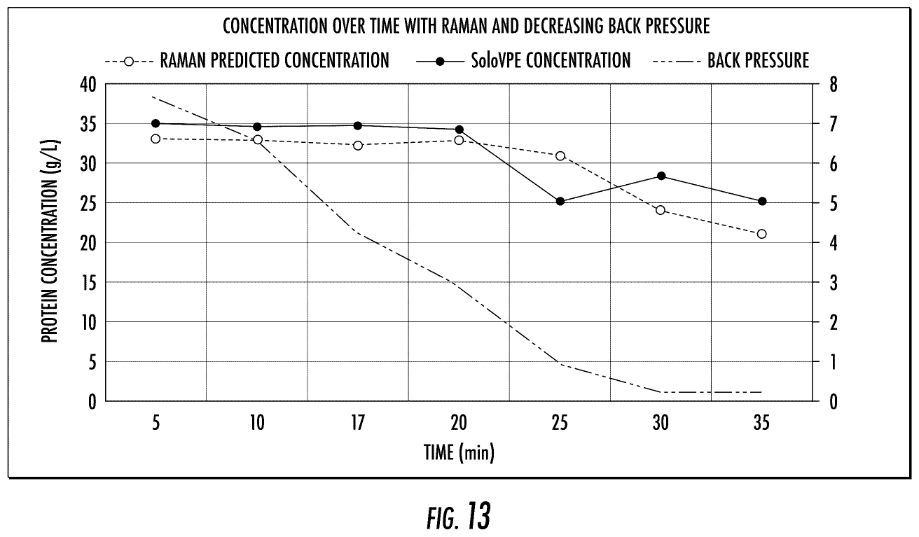

Method: Two SPTFF capsules in series were operated in recirculation mode with permeates and retentate returning to the feed while mixing to save material and maintain a consistent feed concentration. Retentate return was sampled approximately every 5-10 minutes for each run while Raman continuously collected data. For run 1 (FIG. 12) back pressure was controlled to 8 psi using an Equilibar diaphragm valve and compressed air with a pressure regulator. Raman predicted protein concentration was used to monitor continuously as well as indicate if the protein concentration target of 2× was met. As the run progressed, the pump speed was reduced to evaluate the effect of reducing the flow rate on the protein concentration (both Raman predicted and the SoloVPE® concentration). For run 2 (FIG. 13), back pressure was gradually removed to assess the impact on protein concentration.

Results: As Raman and the offline measurements showed the 2× concentration target was not met, the pump speed and feed flux was decreased, resulting in a gradual increase in protein concentration as reflected in the shaded regions in FIG. 12 graph reading from left to right. FIG. 13 shows the results of decreasing the applied retentate back pressure over time on concentration as monitored by Raman. As the back pressure was slowly decreased from 8 to 0 psi, the protein concentration decreased from about 35 g/L as was achieved at 8 psi to a concentration of 25 g/L at 0 psi back pressure which falls below the 2× protein concentration target. Table 7 summarizes run 1 and run 2 protein concentration as predicted by Raman models compared to the offline measurement by SoloVPE®.

TABLE 7
Raman predicted concentration compared to offline protein
concentration measurement by SoloVPE ®.

| | | Raman | Offline SoloVPE | |
|---|---|---|---|---|
| | | Predicted | Protein | |
| | Time | Concentration | Concentration | Offset |
| Run | (min) | (g/L) | (g/L) | (%) |
| 1 | 15 | 22.6 | 27.1 | 16.6 |
| | 20 | 25 | 27 | 7.4 |
| | 25 | 24.7 | 27.1 | 8.9 |
| | 30 | 22.9 | 27.1 | 15.5 |
| | 35 | NR | 27.1 | N/A |
| | 40 | 23.8 | 27.9 | 14.7 |
| | 45 | 23.8 | 28.1 | 15.3 |
| | 50 | 24.2 | 28.6 | 15.4 |
| | 55 | 24.3 | 28.7 | 15.3 |
| | 60 | 26.9 | 30.1 | 10.6 |
| | 65 | 25.8 | 30.4 | 15.1 |
| | 70 | 28.9 | 33 | 12.4 |
| | 75 | 29.1 | 32.9 | 11.6 |
| | 80 | 34.3 | 34.9 | 1.7 |
| | 85 | 35.5 | 34.7 | −2.3 |
| 2 | 5 | 33 | 35.1 | 6.0 |
| | 10 | 32.9 | 34.7 | 5.2 |
| | 17 | 32.4 | 34.8 | 6.9 |
| | 20 | 32.9 | 34.4 | 4.4 |
| | 25 | 31 | 25.2 | −23.0 |
| | 30 | 24 | 28.4 | 15.5 |
| | 35 | 21 | 25.2 | 16.7 |

The percent (%) offset between the two measurements is recorded as a measure of how different the Raman measurement was compared to the offline SoloVPE® measurement. A goal of offset of 6% between the two has been established and the results suggest that more fine tuning of the Raman model will be required to improve accuracy and meet this goal. However, this experiment confirmed that Raman could be used to monitor trends in protein concentration during SPTFF and even control pump speeds to bring the protein concentration closer to the goal.

### Example 10

### Exemplary Antibodies and Other Proteins for Use According to the Inventions

Preferred antibodies, including bispecific antibodies, can be selected from an anti-Programmed Cell Death 1 antibody (for example, an anti-PD1 antibody as described in U.S. Pat. No. 9,987,500B2), an anti-Programmed Cell Death Ligand-1 (for example, an anti-PD-L1 antibody as described in in U.S. Pat. No. 9,938,345B2), an anti-DII4 antibody, an anti-Angiopoetin-2 antibody (for example, an anti-ANG2 antibody as described in U.S. Pat. No. 9,402,898), an anti-Angiopoetin-Like 3 antibody (for example, an anti-AngPtl3 antibody as described in U.S. Pat. No. 9,018,356), an anti-platelet derived growth factor receptor antibody (for example, an anti-PDGFR antibody as described in U.S. Pat. No. 9,265,827), an anti-Erb3 antibody, an anti-Prolactin Receptor antibody (for example, anti-PRLR antibody as described in U.S. Pat. No. 9,302,015), an anti-Complement 5 antibody (for example, an 25 anti-C5 antibody as described in U.S. Pat. No. 9,795,121B2), an anti-TNF antibody, an anti-epidermal growth factor receptor antibody (for example, an anti-EGFR antibody as described in U.S. Pat. No. 9,132,192 or an anti-EGFRvIII antibody as described in U.S. Pat. No. 9,475,875B2), an anti-Proprotein Convertase Subtilisin Kexin-9 antibody (for example, an anti-PCSK9 antibody as described in U.S. Pat. No. 8,062,640 or U.S. Pat. No. 9,540,449B2), an anti-Growth And Differentiation Factor-8 antibody (for example, an anti-GDF8 antibody, also known as anti-myostatin antibody, as described in U.S. Pat. Nos. 8,871,209 or 9,260,515), an anti-Glucagon Receptor (for example, anti-GCGR antibody as described in U.S. Pat. Nos. 9,587,029B2 or 9,657,099B2), an anti-VEGF antibody, an anti-IL1R antibody, an interleukin 4 receptor antibody (e.g., an anti-IL4R antibody as described in U.S. Pat. Nos. 12,162,943B2, 8,735,095 or 8,945,559), an anti-interleukin 6 receptor antibody (for example, an anti-IL6R antibody as described in U.S. Pat. Nos. 7,582,298, 8,043,617 or 9,173,880), an anti-IL1 antibody, an anti-IL2 antibody, an anti-IL3 antibody, an anti-IL4 antibody, an anti-IL5 antibody, an anti-IL6 antibody, an anti-IL7 antibody, an anti-interleukin 33 (for example, anti-IL33 antibody as described in U.S. Pat. Nos. 9,453,072B2 or 9,637,535B2), an anti-Respiratory syncytial virus antibody (for example, anti-RSV antibody as described in U.S. Pat. No. 9,447,173B2), an anti-Cluster of differentiation 3 (for example, an anti-CD3 antibody, as described in U.S. Pat. Nos. 9,657,102B2 and 10,550,193B2, and in U.S. Pat. Appln. Pub. No. US20210253701A1), an anti-Cluster of differentiation 20 (for example, an anti-CD20 antibody as described in U.S. Pat. Nos. 9,657,102B2 and 10,550,193B2, and in U.S. Pat. No. 7,879,984), an anti-CD19 antibody, an anti-CD28 antibody, an anti-Cluster of Differentiation 48 (for example, anti-CD48 antibody as described in U.S. Pat. No. 9,228,014), an anti-Fel d1 antibody (for example, as described in U.S. Pat. No. 9,079,948), an anti-Middle East Respiratory Syndrome virus (for example, an anti-MERS antibody as described in U.S. Pat. No. 9,718,872B2), an anti-Ebola virus antibody (for example, as described in U.S. Pat. No. 9,771,414B2), an anti-Zika virus antibody, an anti-Lymphocyte Activation Gene 3 antibody (for example, an anti-LAG3 antibody, or an anti-CD223 antibody), an anti-Nerve Growth Factor antibody (for example, an anti-NGF antibody as described in U.S. Pat. Appln. Pub. No. US2016/0017029 and U.S. Pat. Nos. 8,309,088 and 9,353,176) and an anti-Activin A antibody. In some embodiments, the bispecific antibody is selected from the group consisting of an anti-CD3×anti-CD20 bispecific antibody (as described in U.S. Pat. Nos. 9,657,102B2 and 10,550,193B2), an anti-CD3×anti-Mucin 16 bispecific antibody (for example, an anti-CD3×anti-Muc16 bispecific antibody), and an anti-CD3×anti-Prostate-specific membrane antigen bispecific antibody (for example, an anti-CD3×anti-PSMA bispecific antibody). See also U.S. Pat. No. 11,054,389B2. Also included are a Met×Met antibody, an agonist antibody to NPR1, an LEPR agonist antibody, a BCMA×CD3 antibody, a MUC16×CD28 antibody, a GITR antibody, an IL-2Rg antibody, an EGFR×CD28 antibody, a Factor XI antibody, antibodies against SARS-CoC-2 variants, a Fel d 1 multi-antibody therapy, a Bet v 1 multi-antibody therapy, an anti-PD1 antibody, an anti-PDL-1 antibody, an anti-DII4 antibody, an anti-ANG2 antibody, an anti-AngPtl3 antibody, an anti-PDGFR antibody, an anti-Erb3 antibody, an anti-PRLR antibody, an anti-TNF antibody, an anti-EGFR antibody, an anti-PCSK9 antibody, an anti-GDF8 antibody, an anti-GCGR antibody, an anti-VEGF antibody, an anti-IL1R antibody, an anti-IL4R antibody, an anti-IL6R antibody, an anti-IL1 antibody, an anti-IL2 antibody, an anti-IL3 antibody, an anti-IL4 antibody, an anti-IL5 antibody, an anti-IL6 antibody, an anti-IL7 antibody, an anti-RSV antibody, an anti-NGF antibody, an anti-CD3 antibody, an anti-CD20 antibody, an anti-CD19 antibody, an anti-CD28 antibody, an anti-CD48 antibody, an anti-CD3/anti-CD20 bispecific antibody, an anti-CD3/anti-MUC16 bispecific antibody, an anti-CD3/anti-PSMA bispecific antibody, a PDGF-b antagonist; an anti-NPR1 antibody; an anti-Factor XII antibody; an anti-TMPRSS6 antibody; an anti-Factor XI antibody; a Multi antibody to Fel d 1; a Multi antibody to Bet v 1; a bispecific anti-PSMA, an anti-CD3 antibody; an anti-PD1-IL2Ra antibody; a bispecific anti-CD38 and anti-CD28 antibody; a bispecific anti-CD22 and anti-CD28 antibody; a bispecific anti-MUC16 and anti-CD28 antibody; an anti-EGFR antibody; an anti-CD28 antibody; PDGF-b antagonist; and anti-Myostatin (GDF8) antibody. Additional preferred bispecific antibodies and bispecific preparations include Dupilumab/Linvoseltamab (IL-4R/BCMA and CD3). Still other preferred antibodies, including bispecific antibodies, can be Alirocumab, Atoltimab, Casirivimab, Cemiplimab, Cemiplimab-rwlc, Dupilumab, Evinacumab, Evinacumab-dgnb, Fasinumab, Fianlimab, Garetsomab, Linvoseltamab, Linvoseltamab-gcpt, Maftivimab, Mibavademab, Odesivimab, Odesivimab-ebgn Pozelimab, Pozelimab-bbfg, Rinucumab, Sarilumab, Trevogrumab, Ubamatamab, Nezastomig, Vonsetamig, and Davutamig.

Preferred Receptor Fc-fusion proteins are also referred to as “traps,” “trap molecules” or “trap proteins.” For example, such trap proteins include an IL-1 trap (for example, Rilonacept, see U.S. Pat. No. 6,927,044), or a VEGF Trap (for example, Aflibercept, See U.S. Pat. Nos. 7,087,411 and 7,279,159). There also are proteins that lack Fc portions, such as recombinantly produced enzymes and mini-traps. Mini-traps are trap proteins that use a multimerizing component (MC) instead of an Fc portion and are disclosed in U.S. Pat. Nos. 7,279,159 and 7,087,411. Derivatives, components, domains, chains and fragments of the above also are included.

Other antibodies include Odronextamab, Itepekimab, Nesvacumab, Abciximab, Adalimumab, Adalimumab-atto, Ado-trastuzumab, Alemtuzumab, Atezolizumab, Avelumab, Basiliximab, Belimumab, Benralizumab, Bevacizumab, Bezlotoxumab, Blinatumomab, Brentuximab vedotin, Brodalumab, Canakinumab, Capromab pendetide, Certolizumab pegol, Cetuximab, Denosumab, Dinutuximab, Durvalumab, Eculizumab, Elotuzumab, Emicizumab-kxwh, Evolocumab, Golimumab, Guselkumab, Ibritumomab tiuxetan, Idarucizumab, Infliximab, Infliximab-abda, Infliximab-dyyb, Ipilimumab, Ixekizumab, Mepolizumab, Necitumumab, Nivolumab, Obiltoxaximab, Obinutuzumab, Ocrelizumab, Oatumumab, Ofatumumab, Olaratumab, Omalizumab, Panitumumab, Pembrolizumab (lambrolizumab), Pertuzumab, Ramucirumab, Ranibizumab, Ravulizumab-cwvz, Raxibacumab, Reslizumab, Rituximab, Secukinumab, Siltuximab, Tocilizumab, Ustekinumab, Vedolizumab, anrukinzumab, atczolizumab, atlizumab, alacizumab pegol, etrolizumab, efalizumab, obexelimab, gantenerumab, inclacumab, brolucizumab, batoclimab, cedelizumab, crenezumab, Enoblituzumab, glycooptimized trastuzumab-GEX, Trastuzumab emtansine HER2, gemtuzumab, gemtuzumab ozogamicin, nimotuzumab, palivizumab, baciliximab, daclizumab, natalizumab, otelixizumab, teplizumab, epratuzumab, briakinumab, abagovomab, adecatumumab, afutuzumab, altumomab pentetate, amatuximab, anatumomab mafenatox, anetumab ravtansine, apolizumab, apomab, arcitumomab, ascrinvacumab, bavituximab, bectumomab, besilesomab, bivatuzumab mertansine, Brontictuzumab, cantuzumab mertansine, cantuzumab ravtansine, carlumab, catumaxomab, cBR-doxorubicin immunoconjugate, citatuzumab bogatox, cixutumumab, clenoliximab, clivatuzumab tetraxetan, codrituzumab, coltuximab ravtansine, conatumumab, dacetuzumab, dalotuzumab, dalotuzumab, daratumumab, demcizumab, denintuzumab mafodotin, depatuxizumab, derlotuximab, detumomab, drozitumab, duligotumab, duligotuzumab, dusigitumab, ecromeximab, edrecolomab, elgemtumab, elsilimomab, emactuzumab, emibetuzumab, emibetuzumab, enavatuzumab, enfortumab vedotin, enoticumab, ensituximab, epitumomab cituxetan, ertumaxomab, etaracizumab, faralimomab, farletuzumab, FBTA (CD20×CD3), ficlatuzumab, figitumumab, flanvotumab, fresolimumab, futuximab, galiximab, gantiumab, gatipotuzumab, girentuximab, glembatumumab vedotin, icrucumab, igovomab, IMAB362 (CLDN18.2), imgatuzumab, indatuximab ravtansine, indusatumab vedotin, inebilizumab, inotuzumab ozogamicin, intetumumab, iratumumab, isatuximab, ipritumomab labetuzumab, lampalizumab, lenzilumab, lexatumumab, lifastuzumab vedotin, lilotomab satetraxetan, lebrikizumab, ligelizumab, lintuzumab, lirilumab, loncastuximab tesirine, lonvastuximab, lorvotuzumab mertansine, lucatumumab, lumiliximab, lumretuzumab, mapatumumab, margetuximab, matuzumab, milatuzumab, minretumomab, mirvetuximab soravtansine, mitumomab, mogamulizumab, moxetumomab pasudotox, muromonab-CD3, nacolomab tafenatox, naptumomab estafenatox, narnatumab, nofetumomab merpentan, binutuzumab, ocaratuzumab, onartuzumab, ontuxizumab, oportuzumab monatox, oregovomab, otlertuzumab, pankomab, parsatuzumab, pasotuxizumab, patritumab, pemtumomab, pidilizumab, pinatuzumab vedotin, pintumomab, polatuzumab vedotin, quilizumab, racotumomab, radretumab, rilotumumab, robatumumab, romosozumab, rontalizumab, sacituzumab govitecan, samalizumab, satralizumab, satumomab pendetide, seribantumab, seribantumab, SGN-CDA (CD19), SGN-CDA (CD33), sibrotuzumab, simtuzumab, siplizumab, sofituzumab vedotin, solitomab, sonepcizumab, tabalumab, tacatuzumab tetraxetan, tafasitamab, taplitumomab paptox, tarextumab, tenatumomab, teneliximab, teprotumumab, tetulomab, TGN (CD28), tigatuzumab, Timigutuzumab, tomuzotuximab, tositumomab, tovetumab (CD140a), tovetumab (PDGFRα), TRBS (GD2), tralokinumab, Tremelimumab, Ticilimumab, tucotuzumab celmoleukin, ublituximab (MS4A1), ublituximab (CD20), ulocuplumab, urelumab, vandortuzumab vedotin, vantictumab, vanucizumab, varlilumab, veltuzumab, vesencumab, visilizumab, volociximab, vorsetuzumab, votumumab, zalutumumab, zanolimumab, zatuximab, and ziralimumab.

Other antibodies and targets can be palivizumab (RSV); daclizumab (IL-2); gemtuzumab (CD33); natalizumab (VLA-4); teplizumab (CD3); epratuzumab (CD22); briakinumab (IL-12, 23); HuM291 (CD3 fc receptor); HeFi-1, CD30); MDX-060 (CD30); MDX-1401 (CD30); SGN-30 (CD30); HCD122 (CD40); SGN-40 (CD40); MDX-1411 (CD70); hLL1 (EPB-1) (CD74.38); MT293 (TRC093/D93) (cleaved collagen); HuLuc63 (CS1); AMG-655 (DR5); CS-1008 (DR5); IMC-11F8, (EGFR); CDX-110 (EGFRvIII); MORAb-003 (folate receptor a); KW-2871 (ganglioside GD3); MORAb-009 (GP-9); CDX-1307 (MDX-1307) (hCGb); AMG-479 (IGF-1 R); anti-IGF-1R R1507 (IGF1-R); CP 751871 (IGF1-R); IMC-A12 (IGF1-R); B11B022 (IGF-1 R); Mik-beta-1 (IL-2Rb (CD122)); CNTO 328 (IL6); Anti-KIR (1-7F9) (Killer cell Ig-like Receptor (KIR)); Hu3S193 (Lewis (y)); hCBE-11 (LTOR); HuHMFG1 (MUC1); RAV12 (N-linked carbohydrate epitope); CAL (parathyroid hormone-related protein (PTH-rP)); CT-011 (PD1); MDX-1106 (ono-4538) (PD1); MAb CT-O11 (PD1); IMC-3G3 (PDGFRα); huJ591 (PSMA); muJ591 (PSMA,); GC1008 (TGFb (pan) inhibitor (IgG4)); A27.15 (transferrin receptor); E2.3 (transferrin receptor); HuMV833 (VEGF); IMC-18F1 (VEGFR1); IMC-1121 (VEGFR2); GPC3 monoclonal antibody; AER-001, ABT-308 (also referred to as humanized 13C5.5 antibody); RG7636 (anti-ETBR); RG7458 (anti-MUC16); RG7599 (anti-NaPi2b); MPDL3280A (anti-PD-L1); RG7450 (anti-STEAP1); and GDC-0199 (anti-Bcl-2).

Other antibodies or tumor target binding proteins include (for example, TCR domains) include, but are not limited to, those that bind the following antigens (the cancer indications represent non-limiting examples): aminopeptidase N (CD13), annexin Al, CA125 (ovarian cancers), CA15-3 (carcinomas), CA19-9 (carcinomas), L6 (carcinomas), Lewis Y (carcinomas), Lewis X (carcinomas), alpha fetoprotein (carcinomas), CA242 (colorectal cancers), placental alkaline phosphatase (carcinomas), prostate specific antigen (prostate), prostatic acid phosphatase (prostate), epidermal growth factor (carcinomas), CD2 (Hodgkin's disease, NHL lymphoma, multiple myeloma), CD3 epsilon (T cell lymphoma, lung, breast, gastric, ovarian cancers, autoimmune diseases, malignant ascites), CD19 (B cell malignancies), CD20 (non-Hodgkin's lymphoma, B-cell neoplasms, autoimmune diseases), CD21 (B-cell lymphoma), CD22 (leukemia, lymphoma, multiple myeloma, SLE), CD30 (Hodgkin's lymphoma), CD33 (leukemia, autoimmune diseases), CD38 (multiple myeloma), CD40 (lymphoma, multiple myeloma, leukemia (CLL)), CD51 (metastatic melanoma, sarcoma), CD52 (leukemia), CD56 (small cell lung cancers, ovarian cancer, Merkel cell carcinoma, and the liquid tumor, multiple myeloma), CD66e (carcinomas), CD70 (metastatic renal cell carcinoma and non-Hodgkin's lymphoma), CD74 (multiple myeloma), CD80 (lymphoma), CD98 (carcinomas), CD123 (leukemia), mucin (carcinomas), CD221 (solid tumors), CD227 (breast, ovarian cancers), CD262 (NSCLC and other cancers), CD309 (ovarian cancers), CD326 (solid tumors), CEACAM3 (colorectal, gastric cancers), CEACAM5 (CEA, CD66e) (breast, colorectal and lung cancers), DLL4 (A-like-4), EGFR (various cancers), CTLA4 (melanoma), CXCR4 (CD 184, heme-oncology, solid tumors), Endoglin (CD 105, solid tumors), EPCAM (epithelial cell adhesion molecule, bladder, head, neck, colon, NHL prostate, and ovarian cancers), ERBB2 (lung, breast, prostate cancers), FCGR1 (autoimmune diseases), FOLR (folate receptor, ovarian cancers), FGFR (carcinomas), GD2 ganglioside (carcinomas), G-28 (a cell surface antigen glycolipid, melanoma), GD3 idiotype (carcinomas), heat shock proteins (carcinomas), HER1 (lung, stomach cancers), HER2 (breast, lung and ovarian cancers), HLA-DR10 (NHL), HLA-DRB (NHL, B cell leukemia), human chorionic gonadotropin (carcinomas), IGF1R (solid tumors, blood cancers), IL-2 receptor (T-cell leukemia and lymphomas), IL-6R (multiple myeloma, RA, Castleman's disease, IL6 dependent tumors), integrins (avP3, α5β1, 0604, al 103, 0505, avP5, for various cancers), MAGE-1 (carcinomas), MAGE-2 (carcinomas), MAGE-3 (carcinomas), MAGE 4 (carcinomas), anti-transferrin receptor (carcinomas), p97 (melanoma), MS4A1 (membrane-spanning 4-domains subfamily A member 1, Non-Hodgkin's B cell lymphoma, leukemia), MUC1 (breast, ovarian, cervix, bronchus and gastrointestinal cancer), MUC16 (CA125) (ovarian cancers), CEA (colorectal cancer), gp100 (melanoma), MARTI (melanoma), MPG (melanoma), MS4A1 (membrane-spanning 4-domains subfamily A, small cell lung cancers, NHL), nucleolin, Neu oncogene product (carcinomas), P21 (carcinomas), nectin-4 (carcinomas), paratope of anti-(N-glycolylneuraminic acid, breast, melanoma cancers), PLAP-like testicular alkaline phosphatase (ovarian, testicular cancers), PSMA (prostate tumors), PSA (prostate), ROB04, TAG 72 (tumour associated glycoprotein 72, AML, gastric, colorectal, ovarian cancers), T cell transmembrane protein (cancers), Tie (CD202b), tissue factor, TNFRSF10B (tumor necrosis factor receptor superfamily member 10B, carcinomas), TNFRSF13B (tumor necrosis factor receptor superfamily member 13B, multiple myeloma, NHL, other cancers, RA and SLE), TPBG (trophoblast glycoprotein, renal cell carcinoma), TRAIL-R1 (tumor necrosis apoptosis inducing ligand receptor 1, lymphoma, NHL, colorectal, lung cancers), VCAM-1 (CD106, Melanoma), VEGF, VEGF-A, VEGF-2 (CD309) (various cancers).

It is to be understood that the description, specific examples, and data, while indicating exemplary embodiments, are given by way of illustration, and are not intended to limit the present inventions. Various changes and modifications within the present inventions, including combining embodiments in whole and in part, will become apparent to the skilled artisan from the discussion, disclosure and data contained herein, and thus are considered part of the inventions.

What is claimed is:

1. A method of purifying a therapeutic protein from a high-titer cell culture, wherein the method comprises the steps of:
(a) subjecting the high-titer cell culture to affinity capture chromatography (ACX) and viral inactivation (VI) to generate a VI pool;
(b) subjecting the VI pool to VI pool filtration (VIPF) system connected to a single pass tangential flow filtration (SPTFF) system, thereby concentrating the pool volume, and generating a VIPF-SPTFF pool;
(c) subjecting VIPF-SPTFF pool to a chromatography 1 column and then a chromatography 2 column to generate a pool to load on a virus retentive filtration (VRF) system to generate a VRF pool, wherein the chromatography 1 column and the chromatography 2 column are different; and
(d) subjecting the VRF pool to ultrafiltration and/or diafiltration (UF/DF), thereby obtaining the therapeutic protein from the high-titer cell culture.

2. The method according to claim 1, wherein step (c) further comprises a chromatography 3 column that is different and downstream from the chromatography 1 column and the chromatography 2 column, wherein the pool from the chromatography 3 column is loaded on the VRF system to generate the VRF pool.

3. The method according to claim 1, wherein the chromatography 1 column is selected from the group consisting of an anion exchange chromatography (AEX) column, a cation exchange chromatography (CEX) column, a hydrophobic interaction chromatography (HIC) column, and the chromatography 2 column is selected from the group consisting of an AEX column, a CEX column, and a HIC column.

4. The method according to claim 2, wherein the chromatography 1 column is selected from the group consisting of an anion exchange chromatography (AEX) column, a cation exchange chromatography (CEX) column, and a hydrophobic interaction chromatography (HIC) column, the chromatography 2 column is selected from the group consisting of an AEX column, a CEX column, and a HIC column, and the chromatography 3 column is selected from the group consisting of an AEX column, a CEX column, and a HIC column.

5. A method of purifying a therapeutic protein from a high-titer cell culture, wherein the method comprises the steps of:
(a) subjecting the high-titer cell culture to affinity capture chromatography (ACX) and viral inactivation (VI) to generate a VI pool;
(b) subjecting the VI pool to VI pool filtration (VIPF) system serial connected to a single pass tangential flow filtration (SPTFF) system, thereby concentrating the pool volume, and generating a VIPF-SPTFF pool;
(c) subjecting the VIPF-SPTFF pool to anion exchange chromatography (AEX) column to generate an AEX pool;
(d) subjecting the AEX pool to a hydrophobic interaction chromatography (HIC) column to generate an HIC pool;
(e) subjecting the HIC pool to a virus retentive filtration (VRF) system to generate a VRF pool; and
(f) subjecting the VRF pool to ultrafiltration and/or diafiltration (UF/DF), thereby obtaining the therapeutic protein from the high-titer cell culture.

6. The method according to claim 5, wherein the method comprises at least one buffering agent selected from the group consisting of Bis-Tris-Propane (BTP), Bis-Tris, Tris, Glycine, Bicine, Tricine, Acetate, Borate, Citrate, Carbonate, Phosphate, Formate, Sulfate, Succinic acid, Sulfonic acid, Diethanolamine, and Imidazole.

7. The method according to claim 5, wherein the buffer comprises about 1 mM to about 50 mM of the buffering agent, about 50 mM to about 500 mM salt, about 0.001% (w/v) to about 0.3% (w/v) non-ionic surfactant, and a pH of about 3.5 to about 11.0.

8. The method according to claim 5, wherein flow rates are the same across the VIPF filtration and the SPTFF system.

9. The method according to claim 5, wherein the concentrating the pool volume and volume reduction by SPTFF are controlled by controlling the overall filtration and SPTFF process flow rate or by SPTFF back pressure.

10. The method according to claim 5, wherein the high-titer cell culture having a feed protein concentration of at least 5 g/L to about 50 g/L.

11.-15. (canceled)

16. The method according to claim 5, wherein the therapeutic protein is a monoclonal antibody.

17. A therapeutic protein obtained from a high-titer cell culture, wherein the therapeutic protein is purified by a method comprising the steps of:
(a) subjecting the high-titer cell culture to affinity capture chromatography and viral inactivation (VI) to generate a VI pool;
(b) subjecting the VI pool to VI pool filtration (VIPF) system serial connected to a single pass tangential flow filtration (SPTFF) system, thereby concentrating the pool volume, and generating a VIPF-SPTFF pool;
(c) subjecting the VIPF-SPTFF pool to an anion exchange chromatography (AEX) column to generate an AEX pool;
(d) subjecting the AEX pool to a hydrophobic interaction chromatography (HIC) pee column to generate an HIC pool;
(e) subjecting the HIC pool to a virus retentive filtration (VRF) system to generate VRF pool; and
(f) subjecting VRF pool to ultrafiltration and/or diafiltration (UF/DF), thereby obtaining the therapeutic protein from the high-titer cell culture.

18.-26. (canceled)

27. The method according to claim 1, wherein the method provides in-line concentration and volume reduction of the intermediate process pools through SPTFF, wherein the method comprising the steps of: a normal flow filtration process connected in series to SPTFF;
wherein flow rates are the same across the filtration process and the SPTFF process, and
wherein the concentration and volume reduction by SPTFF are controlled by controlling the overall filtration and SPTFF process flow rate or by SPTFF back pressure.

28. The method according to claim 27, wherein the normal flow filtration includes affinity capture chromatography and VI pool filtration (VIPF) system.

29. The method according to claim 27, wherein the filtration process removes various impurities, including host cell proteins, through adsorptive interactions.

30. The method according to claim 27, wherein the final feed concentration is monitored by Raman Spectroscopy (RS), which is used as feedback control.

31. A therapeutic protein obtained from a high-titer cell culture made by the method according to claim 1.

32. A therapeutic protein obtained from a high-titer cell culture made by the method according to claim 5.

33. A therapeutic protein obtained from a high-titer cell culture made by the method according to claim 27.

34. A system for performing the method according to claim 1.

35. A system for performing the method according to claim 5.

36. A system for performing the method according to claim 27.
